SEQUENCE LISTING (17 sequences)

SEQ ID NO: 1 - DNA, 1762 bp, PMWS-PCV-P1
attctgattaccagcaatcagaccccgttggaatggtactcctcaactgctgtcccagctgtagaagctctctatcggaggattacttccttggtattttggaagaatgctacagaacaatccatggaggaagggggccagttcgtcaccctttcccccccatgccctgaatttccatatgaaataaattactgagtcttttttatcacttcgtaatggtttttatttttcatttaggggttaagtggggggtctttaagattaaattctctgaattgtacatacatggttacacggatattgtagtcctggtcgtatttactgttttcgaacgcagtgccgaggcctacgtggtccacatttctagaggtttgtagcctcagccaaagctgattccttttgttatttggttggaagtaatcaatagtggagtcaagaacaggtttgggtgtgaagtaacgggagtggtaggagaagggttgggggattgtatggcgggaggagtagtttacatatgggtcataggttagggcattggcctttggtacaaagttatcatctagaataacagcagtggagcccactcccctatcaccctgggtgatgggggagcagggccagaattcaaccttaacttttcttattctgtagtattcaaagggtatagagattttgttggtcccccctcccgggggaacaaagtcgtcaagcttaaatctcatcatgtccaccgcccacgagggcgttgtgactgtggtacgcttgacagtatatccgaaggtgcgggagaggcgggtgttgaagatgccatttttccttctccaacggtggcgggggtggacgagccaggggcggcggcggaggatctggccaagatggctgcgggggcggtgtcttcttctgcggtaacgcctccttggatacgtcatagctgaaaacgaaagaagtgcgctgtaagtattaccagcgcacttcggcagcggcagcacctcggcagcacctcagcagcaacatgcccagcaagaagaatggaagaagcggaccccaaccacataaaaggtgggtgttcacgctgaataatccttccgaagacgagcgcaataaaatacgggagctcccaatctccctatttgattattttattgttggcgaggagggtaatgaggaaggacgaacacctcacctccaggggttcgctaattttgtgaagaagcaaacttttaataaagtgaagtggtatttgggtgcccgctgccacatcgagaaagccaaaggaactgatcagcagaataaagaatattgcagtaaagaaggcaacttacttattgaatgtggagctcctcgatctcaaggacaacggagtgacctgtctactgctgtgagtaccttgttggagagcgggagtctggtgaccgttgcagagcagcaccctgtaacgtttgtcagaaatttccgcgggctggctgaacttttgaaagtgagcgggaaaatgcagaagcgtgattggaagaccaatgtacacgtcattgtggggccacctgggtgtggtaaaagcaaatgggctgctaattttgcagacccggaaaccacatactggaaaccacctagaaacaagtggtgggatggttaccatggtgaagaagtggttgttattgatgacttttatggctggctgccgtgggatgatctactgagactgtgtgatcgatatccattgactgtagagactaaaggtggaactgtaccttttttggcccgcagt

SEQ ID NO: 2 - DNA, 1768 bp, PMWS-PCV-P2
attctgattaccagcaatcagaccccgttggaatggtactcctcaactgctgtcccagctgtagaagctctctatcggaggattacttccttggtattttggaagaatgctacagaacaatccacggaggaagggggccagttcgtcaccctttcccccccatgccctgaatttccatatgaaataaattactgagtcttttttatcacttcgtaatggtttttattattcatttagggtttaagtggggggtctttaagattaaattctctgaattgtacatacatggttacacggatattgtagtcctggtcgtatttactgttttcgaacgcagtgccgaggcctacgtggtccacatttctagaggtttgtagcctcagccaaagctgattccttttgttatttggttggaagtaatcaatagtggagtcaagaacaggtttgggtgtgaagtaacgggagtggtaggagaagggttgggggattgtatggcgggaggagtagtttacatatgggtcataggttagggctgtgctctttggaaaaaagttatcatctagaataacagcagtggagcccactcccctatcaccctgggtgatgggggagcagggccagaattcaaccttaacctttcttattctgtagtattcaaagggtatagagattttgttggtcccccctcccgggggaacaaagtcgtcaatcgtaaatctcatcatgtccaccgcccaggagggcgttgtgactgtggtagccttgacagtatatccgaaggtgcgggagaggcgggtgttgaagatgccatttttccttctccaacggtagcggtggcgggggtggacgagccaggggcggcggcggaggatctggccaagatggctgcgggggcggtgtcttcttctgcggtaacgcctccttggatacgtcatagctgaaaacgaaagaagtgcgctgtaagtattaccagcgcacttcggcagcggcagcacctcggcagcacctcagcagcaacatgcccagcaagaagaatggaagaagcggaccccaacctcataaaaggtgggtgttcacgctgaataatccttccgaagacgagcgcaagaaaatacgggagctcccaatctccctatttgattattttattgttggcgaggagggtaatgaggaaggacgaacacctcacctccaggggttcgctaattttgtgaagaagcaaacttttaataaagtgaagtggtatttgggtgcccgctgccacatcgagaaagccaaaggaactgatcagcagaataaagaatattgcagtaaagaaggcaacttacttattgaatgtggagctcctcgatctcaaggacaacggagtgacctgtctactgctgtgagtaccttgttggagagcgggagtctggtgaccgttgcagagcagcaccctgtaacgtttgtcagaaatttccgcgggctggctgaacttttgaaagtgagcgggaaaatgcagaagcgtgattggaagaccaatgtacacgtcattgtggggccacctgggtgtggtaaaagcaaatgggctgctaattttgcagacccggaaaccacatactggaaaccacctagaaacaagtggtgggatggttaccatggtgaagaagtggttgttattgatgacttttatggctggctgccgtgggatgatctactgagactgtgtgatcgatatccattgactgtagagactaaaggtggaactgtaccttttttggcccgcagt

SEQ ID NO: 3 - DNA, 1768 bp, PMWS-PCV-P3
attctgattaccagcaatcagaccccgttggaatggtactcctcaactgctgtcccagctgtagaagctctctatcggaggattacttccttggtattttggaagaatgctacagaacaatccacggaggaagggggccagttcgtcaccctttcccccccatgccctgaatttccatatgaaataaattactgagtcttttttatcacttcgtaatggtttttatttttcatttaggggttaagtggggggtctttaagattaaattctctgaattgtacatacatggttacacggatattgtagtcctggtcgtatttactgttttcgaacgcagtgccgaggcctacgtggtccacatttctagaggtttgtagcctcagccaaagctgattccttttgttatttggttggaagtaatcaatagtggagtcaagaacaggtttgggtgtgaagtaacgggagtggtaggagaagggttgggggattgtatggcgggaggagtagtttacatatgggtcataggttagggcattggcctttggtacaaagttatcatctagaataacagcagtggagcccactcccctatcaccctgggtgatgggggagcagggccagaattcaaccttaacttttcttattctgtagtattcaaagggtatagagattttgttggtcccccctcccgggggaacaaagtcgtcaagcttaaatctcatcatgtccaccgcccaggagggcgttgtgactgtggtacgcttgacagtatatccgaaggtgcgggagaggcgggtgttgaagatgccatttttccttctccaacggtagcggtggcgggggtggacgagccaggggcggcggcggaggatctggccaagatggctgcgggggcggtgtcttcttctgcggtaacgcctccttggatacgtcatagctgaaaacgaaagaagtgcgctgtaagtattaccagcgcacttcggcagcggcagcacctcggcagcacctcagcagcaacatgcccagcaagaagaatggaagaagcggaccccaaccacataaaaggtgggtgttcacgctgaataatccttccgaagacgagcgcaagaaaatacgggagctcccaatctccctatttgattattttattgttggcgaggagggtaatgaggaaggacgaacacctcacctccaggggttcgctaattttgtgaagaagcaaactttcaataaagtgaagtggtatttgggtgcccgctgccacatcgagaaagccaaaggaactgatcagcagaataaagaatattgcagtaaagaaggcaacttacttattgaatgtggagctcctcgatctcaaggacaacggagtgacctgtctactgctgtgagtaccttgttggagagcgggagtctggtgaccgttgcagagcagcaccctgtaacgtttgtcagaaatttccgcgggctggctgaacttttgaaagtgagcgggaaaatgcagaagcgtgattggaagaccaatgtacacgtcattgtggggccacctgggtgtggtaaaagcaaatgggctgctaattttgcagacccggaaaccacatactggaaaccacctagaaacaagtggtgggatggttaccatggtgaagaagtggttgttattgatgacttttatggctggctgccgtgggatgatctactgagactgtgtgatcgatatccattgactgtagagactaaaggtggaactgtaccttttttggcccgcagt

SEQ ID NO: 4 - DNA, 1768 bp, PMWS-PCV-P4
attctgattaccagcaatcagaccccgttggaatggtactcctcaactgctgtcccagctgtagaagctctctatcggaggattacttccttggtattttggaagaatgctacagaacaatccacggaggaagggggccagttcgtcaccctttcccccccatgccctgaatttccatatgaaataaattactgagtcttttttatcacttcgtaatggtttttattattcatttagggtttaagtggggggtctttaagattaaattctctgaattgtacatacatggttacacggatattgtagtcctggtcgtatttactgttttcgaacgcagtgccgaggcctacgtggtctacatttctagaggtttgtatcctcatccaaagctgattccttttgttatttggttggaagtaatcaatagtggagtcaagaacaggtttgggtgtgaagtaacgggagtggtaggagaagggttgggggattgtatggcgggaggagtagtttacatatgggtcataggttagggctgaggcctttgttacaaagttatcatctagaataacagcagtggagcccactcccctatcaccctgggtgatgggggagcagggccagaattcaaccttaacttttcttattctgtagtattcaaagggtatagagattttgttggtcccccctcccgggggaacaaagtcgtcaatattaaatctcatcatgtccaccgcccaggagggcgttgtgactgtggtagccttgacagtatatccgaaggtgcgggagaggcgggtgttgaagatgccatttttccttctccaacggtagcggtggcgggggtggacgagccaggggcggcggcggaggatctggccaagatggctgcgggggcggtgtcttcttctgcggtaacgcctccttggatacgtcatagctgaaaacgaaagaagtgcgctgtaagtattaccagcgcacttcggcagcggcagcacctcggcagcacctcagcagcaacatgcccagcaagaagaatggaagaagcggaccccaaccacataaaaggtgggtgttcacgctgaataatccttccgaagacgagcgcaagaaaatacgggagctcccaatctccctatttgattattttattgttggcgaggagggtaatgaggaaggacgaacacctcacctccaggggttcgctaattttgtgaagaagcaaacttttaataaagtgaagtggtatttgggtgcccgctgccacatcgagaaagccaaaggaactgatcagcagaataaagaatattgcagtaaagaaggcaacttacttattgaatgtggagctcctcgatctcaaggacaacggagtgacctgtctactgctgtgagtaccttgttggagagcgggagtctggtgaccgttgcagagcagcaccctgtaacgtttgtcagaaatttccgcgggctggctgaacttttgaaagtgagcgggaaaatgcagaagcgtgattggaagaccaatgtacacgtcattgtggggccacctgggtgtggtaaaagcaaatgggctgctaattttgcagacccggaaaccacatactggaaaccacctagaaacaagtggtgggatggttaccatggtgaagaagtggttgttattgatgacttttatggctggctgccgtgggatgatctactgagactgtgtgatcgatatccattgactgtagagactaaaggtggaactgtaccttttttggcccgcagt

SEQ ID NO: 5 - DNA, 1768 bp, CT-PCV-P5; identical to SEQ ID NO: 3

SEQ ID NO: 6 - DNA, 1768 bp, CT-PCV-P6
attctgattaccagcaatcagaccccgttggaatggtactcctcaactgctgtcccagctgtagaagctctctatcggaggattacttccttggtattttggaagaatgctacagaacaatccacggaggaagggggccagttcgtcaccctttcccccccatgccctgaatttccatatgaaataaattactgagtcttttttatcacttcgtaatggtttttattattcatttagggtttaagtggggggtctttaagattaaattctctgaattgtacatacatggttacacggatattgtagtcctggtcgtatttactgttttcgaacgcagtgccgaggcctacgtggtccacatttctagaggtttgtagcctcagccaaagctgattccttttgttatttggttggaagtaatcaatagtggagtcaagaacaggtttgggtgtgaagtaacgggagtggtaggagaagggttgggggattgtgtggcgggaggagtagtttacatatgggtcataggttagggctgtggcctttggtacaaagttatcatctagaataacagcagtggagcccactcccctatcaccctgggtgatgggggagcagggccagaattcaaccttaacctttcttattctgtagtattcaaagggtatagagattttgttggtcccccctcccgggggaacaaagtcgtcaagcttaaatctcatcatgtccaccgcccaggagggcgttgtgactgtggtacgcttgacagtatatccgaaggtgcgggaaagccgggtgttgaagatgccatttttccttctccaacggtagcggtggcgggggtggacgagccaggggcggcggcggaggatctggccaagatggctgcgggggcggtgtcttcttctgcggtaacgcctccttggatacgtcatagctgaaaacgaaagaagtgcgctgtaagtattaccagcgcacttcggcagcggcagcacctcggcagcacctcagcagcaacatgcccagcaagaagaatggaagaagcggaccccaaccacataaaaggtgggtgttcacgctgaataatccttccgaagacgagcgcaagaaaatacgggagctcccaatctccctatttgattattttattgttggcgaggagggtaatgaggaaggacgaacacctcacctccaggggttcgctaattttgtgaagaagcaaacttttaataaagtgaagtggtatttgggtgcccgctgccacatcgagaaagccaaaggaactgatcagcagaataaagaatattgcagtaaagaaggcaacttacttattgaatgtggagctcctcgatctcaaggacaacggagtgacctgtctactgctgtgagtaccttgttggagagcgggagtctggtgaccgttgcagagcagcaccctgtaacgtttgtcagaaatttccgcgggctggctgaacttttgaaagtgagcgggaaaatgcagaagcgtgattggaagaccaatgtacacgtcattgtggggccacctgggtgtggtaaaagcaaatgggctgctaattttgcagacccggaaaccacatactggaaaccacctagaaacaagtggtgggatggttaccatggtgaagaagtggttgttattgatgacttttatggctggctgccgtgggatgatctactgagactgtgtgatcgatatccattgactgtagagactaaaggtggaactgtaccttttttggcccgcagt

SEQ ID NO: 7 - DNA, 1759 bp, CT-PCV-P7
attttgattaccagcaatcaggccccccaggaatggtactcctcaactgctgtcccagctgtagaagctctctatcggaggattactactttgcaattttggaagaatgctggagaacaatccacggaggtacccgaaggccgatttgaagcagtggacccaccctgtgcccttttcccatataaaataaattactgagtcttttttgttatcacatcgtaatggtttttatttttattcatttagagggtctttcaggataaattctctgaattgtacataaatagtcagccttaccacataattttgggctgtgtctgcattttggagcgcaaagccgaggcctgtgtgctcgacattggtgtgggtatttaaatggagccacagctggtttcttttattatttggctggaaccaatcaattgtttggtccagctcaggtttgggggtgaagtacctggagtggtaggtaaagggttgccttatggtgtggcgggaggagtagttaatataggggtcataggccaggttggtggagggggttacaaagttggcatccaagataacaacagtggaccctacacctctttgattagaggtgatggggtctctggggtaaaattcatatttagcctttctaatacggtagtattggaaaggtaggggtagggggttggtgccgcccgagggggggaggaactggccgatgttgaatctgaggtggttaacattccaagatggctgcgagtgtcctccttttatggtgagtacaaattctctagaaaggcgggaattgaagatacccttctttcggcgccatctgtaacggtttctgaaggcggggtgtaccaaatatggtcttctccggaggatgtttccaagatggctgcgggggcgggtccttcttctgcggtaacgcctccttggccacgtcatcctataaaagtgaaagaagtgcgctgctgtagtattaccagcgcacttcggcagcggcagcacctcggcagcgtcagtgaaaatgcccagcaagaaaagcggcccgcaaccccataagaggtgggtgttcacccttaataatccttccgaggaggagaaaaacaaaatacgggagcttccaatctccctttttgattattttgtttgtggagaggaaggtttggaagagggtagaactcctcacctccaggggtttgcgaattttgctaagaagcagacctttaacaaggtgaagtggtattttggtgcccgctgccacatcgagaaagcgaaaggaaccgaccagcagaataaagaatactgcagtaaagaaggccacatacttatcgagtgtggagctccgcggaaccaggggaagcgcagcgacctgtctactgctgtgagtacccttttggagacggggtctttggtgactgtagccgagcagttccctgtaacgtatgtgagaaatttccgcgggctggctgaacttttgaaagtgagcgggaagatgcagcagcgtgattggaagacagctgtacacgtcatagtgggcccgcccggttgtgggaagagccagtgggcccgtaattttgctgagcctagggacacctactggaagcctagtagaaataagtggtgggatggatatcatggagaagaagttgttgttttggatgatttttatggctggttaccttgggatgatctactgagactgtgtgaccggtatccattgactgtagagactaaagggggtactgttccttttttggcccgcagt

SEQ ID NO: 8 - DNA, 20 bp, Artificial Sequence
  primer
tactcctcaactgctgtccc

SEQ ID NO: 9 - DNA, 20 bp, Artificial Sequence
  primer
tccatcccaccacttatttc

SEQ ID NO: 10 - DNA, 18 bp, Artificial Sequence
  primer
acgctgaataatccttcc

SEQ ID NO: 11 - DNA, 20 bp, Artificial Sequence
  primer
ccaacaaaatctctataccc

SEQ ID NO: 12 - DNA, 18 bp, Artificial Sequence
  primer
attaccagcaatcagacc

SEQ ID NO: 13 - DNA, 18 bp, Artificial Sequence
  primer
aacaaccacttcttcacc

SEQ ID NO: 14 - DNA, 19 bp, Artificial Sequence
  primer
agcagggccagaattcaac

SEQ ID NO: 15 - DNA, 22 bp, Artificial Sequence
  primer
cgtcttcggaaggattattcag

SEQ ID NO: 16 - DNA, 21 bp, Artificial Sequence
  primer
gcctagtagaaataagtggtg

SEQ ID NO: 17 - DNA, 20 bp, Artificial Sequence
  primer
agtaatcctccgatagagag

(19) United States
(12) Patent Application Publication
(10) Pub. No.: US 2004/0071728 A1
(43) Pub. Date: Apr. 15, 2004

(54) Vaccine for congenital tremors in pigs

(76) Inventors: Suresh K Mittal, West Lafayette, IN (US); Gregory W. Stevenson, West Lafayette, IN (US); Jiwon Choi, West Lafayette, IN (US); Matti Kiupel, Lansing, MI (US); Charles L. Kanitz, Lafayette, IN (US)

Correspondence Address: Paul F Fehlner, Darby & Darby, Post Office Box 5257, New York, NY, 10150-5257, US

(21) Appl. No.: 10/311,423

(22) PCT Filed: Jun. 15, 2001
(86) PCT No.: PCT/US01/19220

(51) Int. Cl. C12Q 1/70; C07H 21/04; A61K 39/12; C12N 7/00; C07K 14/005

(52) U.S. Cl. 424/186.1; 435/5; 435/69.1; 435/320.1; 435/235.1; 435/325; 536/23.72; 530/350

(57) ABSTRACT

The invention is based on the cloning of porcine circovirus (PCV) strains from pigs with congenital tremors. These results provide a first step for the development of diagnostic and therapeutic diagnostic method comprises determining whether the pig has been infected by a porcine cirovirus strain of type 1 or type 2. The invention further provides a method for the prevention or treatment of congenital tremors in a pig, which method comprises administering to the pig an effective amount of an immunogenic PCV 1 or PCV 2 polypeptide or of a nucleic acid encoding this polypeptide. Another subject of the invention is the new PCV nucleic acid sequences identified by the inventors and the polypeptides encoded by these sequences, as well as the new PCV isolates and immunogenic preparations thereof.

This application claims priority to U.S. Provisional Application Serial No. 60/211,710, filed Jun. 15, 2000 under 35 U.S.C. § 119(e), which is incorporated herein by reference in its entirety.

### FIELD OF THE INVENTION

The present invention relates to the identification of an association between porcine circovirus (PCV) and congenital tremors in pigs and to related diagnostic and therapeutic compositions and methods. The invention more particularly provides specific congenital tremors associated PCV nucleic acids and polypeptides.

### BACKGROUND OF THE INVENTION

Porcine circovirus (PCV) was initially discovered as a noncytopathic contaminant of PK-1 5, a porcine kidney cell line (Tischer et al., Medizinische Mikrobiologie und Parasitologie 226: 153-167, 1974). This virus was characterized in 1982 (Tischer et al., Nature, 295:64-66,1982) and classified among the circoviridae with the chicken anemia virus (Yuasa et al., Avian Diseases 23: 366, 1979), the psittacine beak and feather disease virus (Pass & Perry, Austrial Veterinary Journal, 61:69-74,1984) and the pigeon circovirus (Woods et al., Journal of Veterinary Diagnostic Investigations: 5:609-612, 1983). Circoviral genome consists of a single copy of circular single-stranded ambisense DNA genome (Lukert et al., Sixth Report of the International Committee on Taxonomy of Viruses, 166-168, 1995). The size of the genome varies between 1.7 and 2.3 kb. Circoviruses are non-enveloped and have icosahedral symmetry. The PCV, derived from the PK-15 cells has first been considered not to be pathogenic. Its complete genome was sequenced (Meehan et al., Journal of General Virology 78:221-227 1997) and ithas been characterized electron microscopically (Stevenson et al., Veterinary Pathology 36:368-378, 1999). PK-15-PCV has never been associated with a naturally occurring disease and experimental inoculation of pigs did not result in clinical disease (Tischer et al., Archives of Virology 91:271-276, 1986; Allan et al., Journal of Comparative Pathology 121: 1-11, 1995).

Phylogenetic analysis of PK-15-PCV, chicken and psittacine animal circoviruses, plant geminiviruses and nanoviruses (previously known as plant circoviruses) classified PK-15-PCV as most closely related to psittacine beak and feather disease virus; both PK-15-PCV and psittacine circovirus shared features with and were intermediate between the 2 plant viral groups (Niagro et al., Archives of Virology 143:1723-1744, 1998). Additional analyses suggested that a predecessor to PK-15-PCV and/or psatticine circovirus originated from a plant nanovirus that infected a vertebrate host and recombined with a vertebrate-infecting RNA virus, most likely a calicivirus (Gibbs & Weiller, Proceedings of the National Academy of Sciences, USA, 96:8022-8027, 1999).

Infection by PCV has been associated with postweaning multisystemic wasting syndrome (PMWS), that is clinically characterized by progressive weight loss, dyspnea, tachypnea and icterus in postweaned pigs (Daft et al, Meeting of the American Association of Veterinary Laboratory Diagnosticians, Little Rock, Ark., USA, p32, 1996; Clark, Proceedings of the 28th Annual Meeting of the American Association of Swine Practitioners, Quebec City, Quebec, pp. 499-501 1997; Kiupel et al., Indiana Veterinary Pathology 35:303-307, 1998; Ellis et al., Canadian Veterinary Journal 39:44-51, 1998; Allan & Ellis, Journal of Veterniary Diagnostic Investigations 12:3-14, 2000). The complete genomic sequences of a number of PCV associated with PMWS are available (Hamel et al., Journal of Virology 72:5262-5267, 1998; Meehan et al., Journal of General Virology 79:2171-2179, 1998; Morozov et al., Journal of Clinical Microbiology 9:2535-2541, 1998; Mankertz el al., Virus Research, 6665-77, 2000; WO 99/18214, WO 99/45956, U.S. Pat. No. 6,217,883). Isolates of PMWS-PCV differ from PK-15-PCV antigenically and genetically (Allan et al., Journal of Veterniary Diagnostic Investigations 10:3-10, 1998; Hamel el al, Journal of Virology 72:5262-5267,1998). These PMWS-associated PCV were thus referred to as PCV2 as opposed to the original PK-15 cell culture isolate referred to as PCV 1 (Meehan et al., Journal of General Virology 79:2171-2179, 1998).

Congenital tremors (CT) in pigs are associated with myelin deficiency and may be caused by genetic abnormalities (Harding et al., Vet Rec 92:527-529, 1973; Patterson et al., J Neurochem 26:481-485, 1976), in-utero trichlorfon toxicity (Knox et al., Nord Veterinaermed 30:538-545, 1978) and in-utero infection with classical swine fever virus (Harding et al., Vet Rec 79:388-390, 1966) or Aujeszky's virus (Mare et al., J Am Vet Med Assoc 164:309-310, 1974). The most common form of CT in North America is transmissible and classified as type A2 (Done et al., Veterinary Annual 16:98-102, 1976). The epidemiology of CT type A2 has been reviewed (Bolin et al., eds. Leman AD, Straw BE, Mengeling W L, D'Allaire S, Taylor DJ, 7th ed., pp.247-249, 1992). Disease occurs in all breeds, is not seasonal, is more common in litters of first parity sows and is frequently associated with the introduction of replacement breeding stock from an outside source (Stromberg et al., Am J Vet Res 19:377-382, 1958). Prevalence among and within affected litters varies from 0-100%. Outbreaks usually last for 1-8 weeks, but the disease rarely may be endemic. Affected pigs exhibit clonic contractions of skeletal muscles of varying severity that usually diminish and resolve by 4 weeks-of-age but that may continue until slaughter-age. Myoclonus abates when pigs are resting and is exacerbated by external stimuli (Christensen et al., Nord Veterinaermed 8:921-943, 1956; Stromberg et al., Am J Vet Res 20: pp. 319-323 and 627-633, 1959). Mortality in affected pigs may be as high as 50% and is caused by an inability to suckle.

The association of a virus with CT type A2 has been known for many years, but the virus strains have so far not been identified. The first studies described an unidentified, approximately 20 nm, cuboidal virus in filtrates from primary kidney cell cultures derived from neonatal pigs with CT type A2 (Kanitz CL: 1972, Myoclonia congenital Studies of the resistance to viral infection of tissue culture cell lines derived from myclonic pigs. PhD dissertation, Purdue University, West Lafayette, Ind.). These studies included intramuscular inoculation of pregnant sows with the cuboidal virus prepared as a filtrate of kidney cell-culture supernatant, which resulted in the birth of litters with congenital tremors. Other researchers purified a virus on cesium chloride gradients from primary kidney cell cultures obtained from a pig with CT type A2 and identified the virus as PCV-based on morphology and indirect immunologic methods (Hines RK: 1994. Porcine circovirus causes congenital tremors type A-11 proved by fulfilling Koch's postulates. PhD dissertation, University of Georgia, Athens, Ga.). Subsequent subcutaneous, intranasal and oral inoculation of pregnant sows in the last third of gestation with this purified virus resulted in the birth of pigs with congenital tremors. PCV was re-isolated from intestinal tissues but not nervous tissues of pigs with CT and not from tissues of normal control pigs derived from sham-inoculated dams. PCV DNA was also found in samples from swine herds with CT, using PCR primers sets designed to amplify a PMWS PCV isolate (G. W. Stevenson et al, IX International Symposium, College Station, Tex., June 1999).

However genetic analysis of PCV isolates associated with CT has not been reported yet. Thus there is a need to confirm the association of PCV with congenital tremors and to precisely identify what types of PCV are involved in the disease. Indeed only such an analysis may allow to produce diagnostic and therapeutic tools against congenital tremors in pigs, as currently no effective diagnostic tests nor vaccines for congenital tremors are available.

### SUMMARY OF THE INVENTION

The invention is based on the cloning of porcine circovirus (PCV) strains from pigs with congenital tremors.

These results provide a first step for the development of diagnostic and therapeutic applications.

Accordingly, the present invention provides a method of diagnosis a pathological cause of congenital tremors in a pig, which method comprises determining whether the pig has been infected by a porcine circovirus strain of type 1 or type 2.

The invention further provides a method for the prevention or treatment of congenital tremors in a pig, which method comprises administering to the pig an effective amount of an immunogenic PCV 1 or PCV 2 polypeptide or of a nucleic acid encoding this polypeptide.

Another subject of the invention is the new PCV nucleic acid sequences identified by the inventors and the polypeptides encoded by these sequences, as well as the new PCV isolates and immunogenic preparations thereof.

Accordingly, the invention relates to an isolated porcine circovirus (PCV), which nucleic acid has a sequence that is identical to a sequence selected from the group consisting of SEQ ID NO. 1 to SEQ ID NO. 7.

The invention further relates to an isolated nucleic acid from porcine circovirus (PCV), which nucleic acid comprises a sequence coding for a circovirus polypeptide having a sequence selected from the group consisting of sequences coded by any of ORF1 to ORF11 of any of the sequences of SEQ ID NO. 1 to SEQ ID NO. 7.

Another subject of the invention is an expression vector comprising this nucleic acid operatively associated with an expression control sequence.

This expression vector may be associated to a pharmaceutically acceptable excipient to form a vaccine, that also is part of the invention.

The invention is further directed to a host cell comprising this expression vector.

The invention also provides a method for producing a PCV protein, which method comprises culturing this host cell under conditions that result in expression of the nucleic acid coding for a circovirus.

The polypeptides comprising the amino acid sequences encoded by any of ORF1 to ORF11 of SEQ ID NO: 1 to NO:7 are also described herein, as well as the antibodies directed against these polypeptides.

A further subject of the invention is a method for culturing a porcine circovirus strain which method comprises introducing a nucleic acid comprising a sequence selected from the group consisting of SEQ ID NO:1 TO SEQ ID NO:6 into a suitable host cell under conditions that result in the production of porcine circovirus particles having a genome that comprises a sequence selected from the group consisting of SEQ ID NO:1 TO SEQ ID NO:6.

The invention also encompasses The isolated PCV strains which have a genome comprising a sequence selected from SEQ ID NO:1 to SEQ ID NO:6.

### DRAWINGS

FIG. 1 is a nucleotide sequence comparison of PMWS-PCV-P1, P2, P3, P4, and CT-PCV-P5, -P6, -P7.

- Indicates the same nucleotide.

• Indicates a deleted nucleotide.

The nucleotide sequence of PMWS-PCV and PK-1 5-PCV were taken from Hamel et al, 1998 and Meehan et al., 1997, respectively.

FIG. 2 is a schematic representation of 11 ORFs of PMWS-PCV-P 1 (FIG. 2A) and CT-PCV-P7 (FIG. 2B). The size of genome of PMWS-PCV-PI and CT-PCV-P7 is 1768 and 1759 nucleotides, respectively. The direction of arrows indicates the orientation of each ORF.

FIG. 3 shows a distance matrix analysis of the full genome of porcine and bovine circovirus isolates. Branch length is proportional to the phylogenetic distance of the isolates. The bar represents 10% difference between two sequences. Unrooted tree; the type 1 strains (CT-P7, PMWS-AF012107, and the PK-15 cell line derived strains) were used as outgroup. Bootstrap values (for 100 data sets) are shown (except in the case of PCV 2-B when the tree topology gained by the full-length alignment has not been confirmed). Virus stains are represented by the caused disease (if known) and the strain name (if available, otherwise by the accession number or the proposed restriction enzyme fragmentation type). Stains (other than ours) and their GenBank accession numbers are as follows. PK-15 cell line derived strains: PK-ISA (U49186), PK-1 5B (Y09921), PK-15C (AF071879); PMWS strains: PMWS-AF012107, P/48121 (Imp.1011 48121, AF055393), P/ISU-31 (AJ223185), P/48285 (Imp.1011 48285, AF055394), P/AF027217, P/ISUVDL (ISUVDL 98-15237, AF147751), (Imp.999 (AF055391), P/Imp.1010 (Imp.1010-Stoon, AF055392), strains without described disease: Tainan (AF 166528), MLTW98 (AF154679), B9 (AF086834),9741 (AF086835),412 450 (AF085695), M226 (AF086836), strains with type names referring to restriction enzyme fragmentation pattern: PCV 2-B (AF112862), PCV 2-C (AF109398), PCV 2-D (AF117753), PCV 2-E (AF109399), bovine circovirus: Bovine CV (AF109397).

FIG. 4 is a protein homology comparison of open reading frame 1 (ORF1) of PMWS- and CT-PCV isolates of the invention with the published sequences of a PWMS-PCV and the PK-15-PCV.

FIG. 5 is a protein homology comparison of open reading frame 2 (ORF2) of PMWS- and CT-PCV isolates of the invention with the published sequences of a PWMS-PCV and the PK-15-PCV.

FIG. 6 is a protein homology comparison of open reading frame 3 (ORF3) of PMWS- and CT-PCV isolates of the invention with the published sequences of a PWMS-PCV and the PK-15-PCV.

FIG. 7 is a protein homology comparison of open reading frame 4 (ORF4) of PMWS- and CT-PCV isolates of the invention with the published sequences of a PWMS-PCV and the PK-15-PCV.

FIG. 8 is a protein homology comparison of open reading frame 5 (ORF5) of PMWS- and CT-PCV isolates of the invention with the published sequences of a PWMS-PCV and the PK-15-PCV.

FIG. 9 is a protein homology comparison of open reading frame 6 (ORF6) of PMWS- and CT-PCV isolates of the invention with the published sequences of a PWMS-PCV and the PK-15-PCV.

FIG. 10 is a protein homology comparison of open reading frame 7 (ORF7) of PMWS- and CT-PCV isolates of the invention with the published sequences of a PWMS-PCV and the PK-15-PCV.

FIG. 11 is a protein homology comparison of open reading frame 8 (ORF8) of PMWS- and CT-PCV isolates of the invention with the published sequences of a PWMS-PCV and the PK-1 5-PCV.

FIG. 12 is a protein homology comparison of open reading frame 9 (ORF9) of PMWS- and CT-PCV isolates of the invention with the published sequences of a PWMS-PCV and the PK-1 5-PCV.

FIG. 13 is a protein homology comparison of open reading frame 10 (ORF10) of PMWS- and CT-PCV isolates of the invention with the published sequences of a PWMS-PCV and the PK-15-PCV.

FIG. 14 is a protein homology comparison of open reading frame 11 (ORF 11) of PMWS- and CT-PCV isolates of the invention with the published sequences of a PWMS-PCV and the PK-15-PCV.

### DETAILED DESCRIPTION OF THE INVENTION

The present invention solves the problem of identifying with certainty the strains of etiological agent for congenital tremors. Cloning and sequencing porcine circovirus (PCV) strains from pigs with congenital tremors and comparing them with PCV strains associated with PMWS have resulted in the development of many useful materials available, such as primers, probes or viral strains. The invention permits cloning strains and culturing virus of known CT pathogenicity.

The invention is based, in part, on the discovery that PCV genomic DNA present in neurons and, to a lesser degree, glial cells in the brain and spinal chord of pigs shared very close sequence similarity, greater than about 95%, to the genomic DNA of a strain associated with postweaning multisystemic wasting syndrome (PMWS). The entire genomes of seven isolates of PCV from pigs with congenital tremors (CT) or PMWS were cloned and sequenced. One isolate was from a neonatal pig with CT type A2 that was isolated in the late 1960s. Two recent PCV isolates were from two affected neonatal pigs from different farms with unrelated outbreaks of CT type A2. Four isolates originated from four different farms from pigs with PMWS. The comparative analysis of the four PMWS-PCVs demonstrated that they share 99% sequence identity with each other, and over 96% with previously sequenced PMWS-PCVS. The two new CT-PCVS, however, shared 99% identity with each other and interestingly, also with the new PMWS-PCV isolates. There were no consistent genomic differences between PMWS and new CT isolates. The old CT-PCV showed 98% identity to PK-I 5-derived PCV strains and demonstrated only 72% identity to the new CT-PCVS. Phylogenetic analysis confirmed that PCV isolates could-be divided into two groups. PCV type I is composed of PK-I 5-PCVs and our old isolate of CT-PCV, and PCV type 2 contains PMWS and our new CT isolates.

Additional work established the tissue distribution and genetic type of PCV in 1-2 day-old pigs with naturally occurring CT type A2 using in-situ hybridization, polymerase chain reaction (PCR) and frozen-tissue-section indirect fluorescent antibody tests. CT affected and clinically normal pigs originating from four Midwestern U.S. farms undergoing outbreaks of CT type A2 were selected. All CT and most normal pigs were infected with PCV. PCV was widely distributed in tissues of infected pigs and was most common in central nervous tissues and liver. In all infected pigs, there were more PCV-infected cells in brain and spinal cord than in non-neural tissues. CT pigs had many more PCV-infected cells in the brain and spinal cord than did clinically normal pigs due to a more diffuse distribution and a larger proportion of infected cells. The cells most commonly infected with PCV in brain and spinal cord were large neurons. In non-neural tissues macrophages were the most frequent cell type infected. PCR tests demonstrated only PCV type 2 and not PCV type 1 in all PCV-infected pigs on all four farms.

The invention if further based on evidence that PCV2 can be transmitted from an infected sow to its litter in utero. PCV2 alone or in combination of a co-factor therefore can be congenitally transmitted.

As noted above, these discoveries resolve ambiguity concerning the etiological relationship of PCV with CT, as well as the relationship between PCV virus that is associated with PMWS (prior work, for example, left open the possibility that another PCV virus strain was associated with CT, if indeed PCV was the etiological agent for CT). Thus, in addition to the novel PCV strains described herein (e.g., PMWS-PCV-P1, PMWS-PCV-P2, PMWS-PCV-P3, PMWS-PCV-P4, CT-PCV-P5, CT-PCV-P6, and CT-PCV-P7), the invention provides diagnostic and therapeutic methods and materials based on these reagents, and identifies PCV, particularly PCV type 2, as a target for diagnostic evaluation and therapeutic, particularly immunological, intervention.

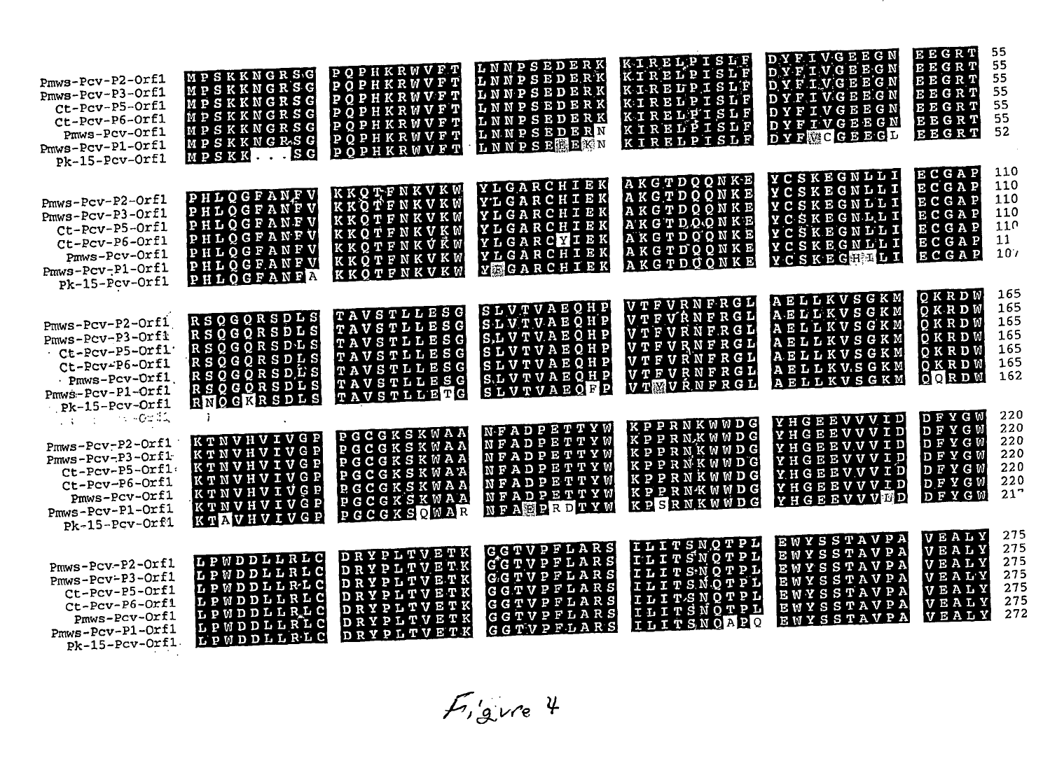

As used herein, the term “PCV” refers specifically to a pig circovirus, e.g., as shown in the distance matrix analysis of the full genome of porcine and bovine circovirus isolates (FIG. 3). In particular, the term PCV means PMWS-PCV-P1 (SEQ ID NO:1), PMWS-PCV-P2 (SEQ ID NO:2), PMWS-PCV-P3 (SEQ ID NO:3), PMWS-PCV-P4 (SEQ ID NO:4), CT-PCV-P5 (SEQ ID NO:5), and CT-PCV-P6 (SEQ ID NO:6). In addition, PCV includes CT-PCV-P7 (SEQ ID NO:7), PK-15 PCV (Meehan et al., 1997), and PMWS-PCV (Hamel et al., 1998).

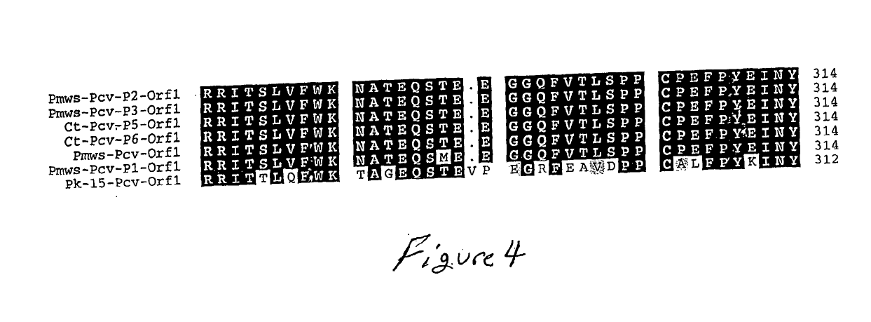

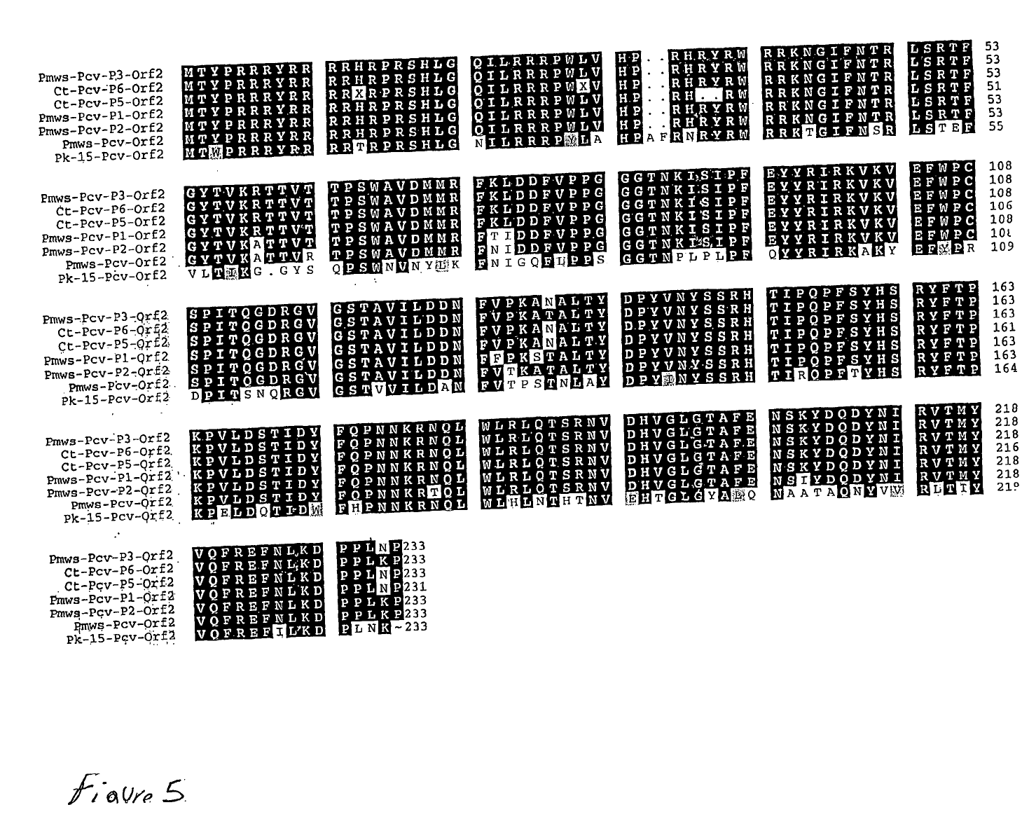

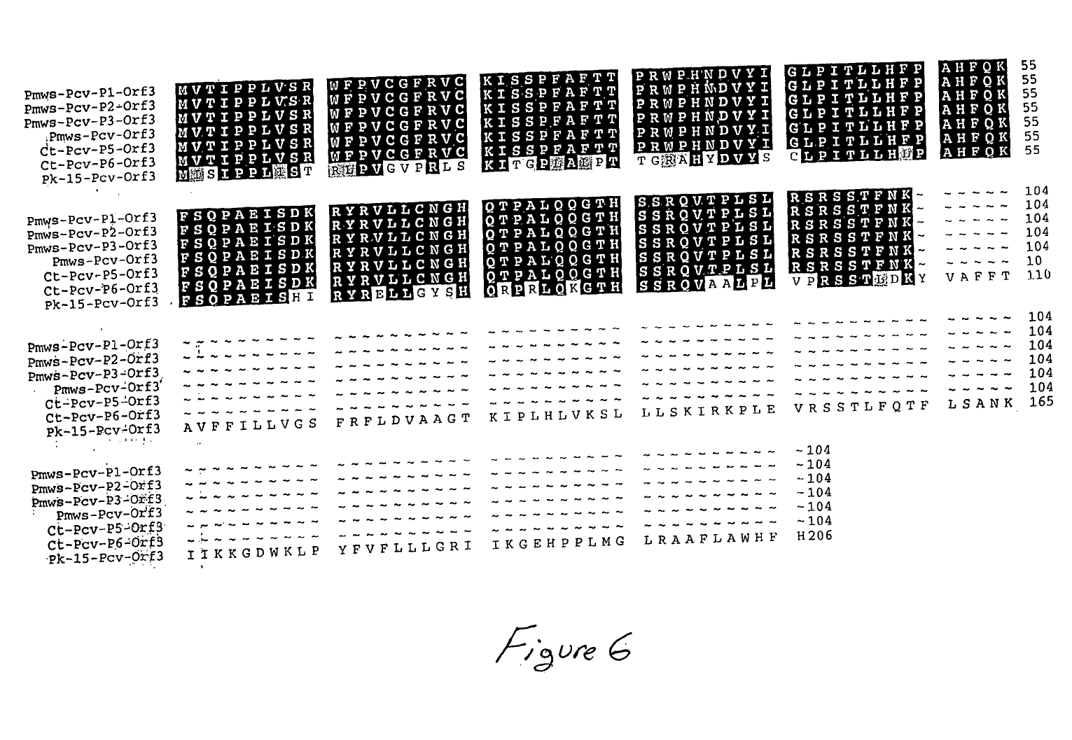

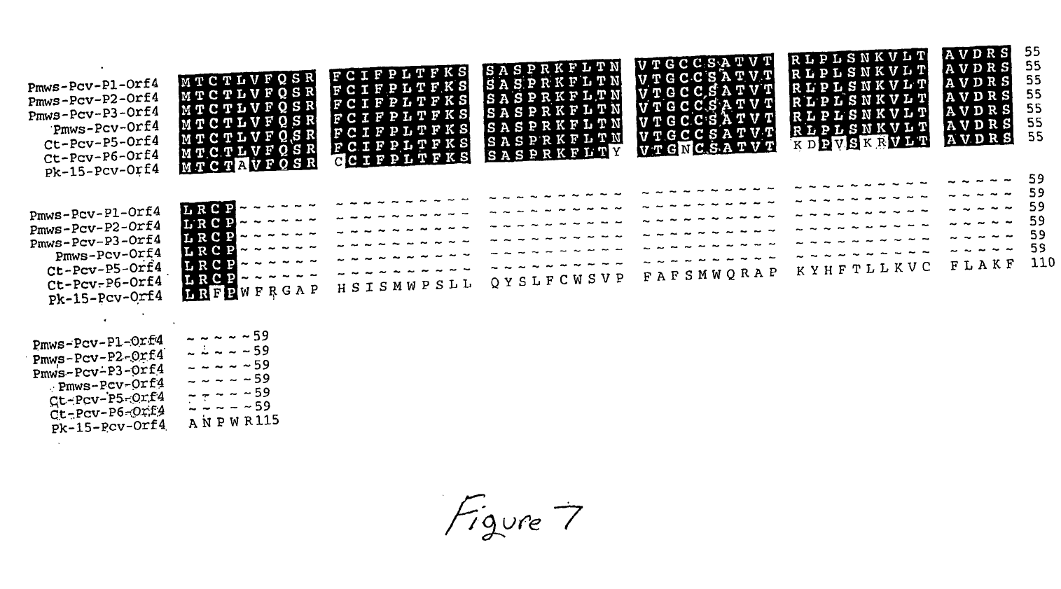

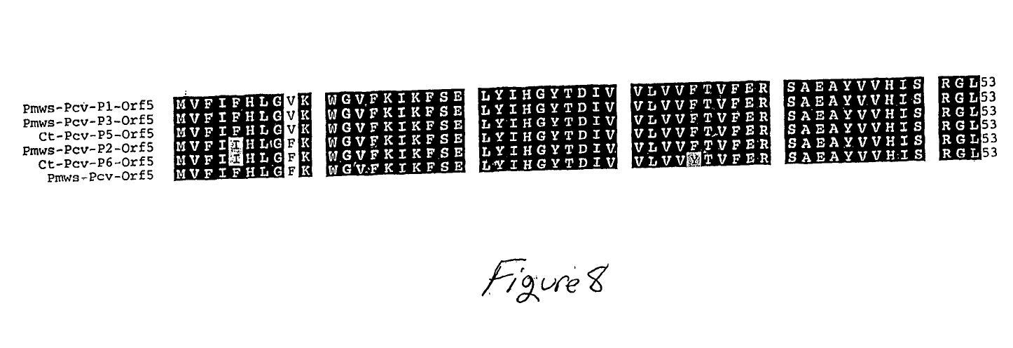

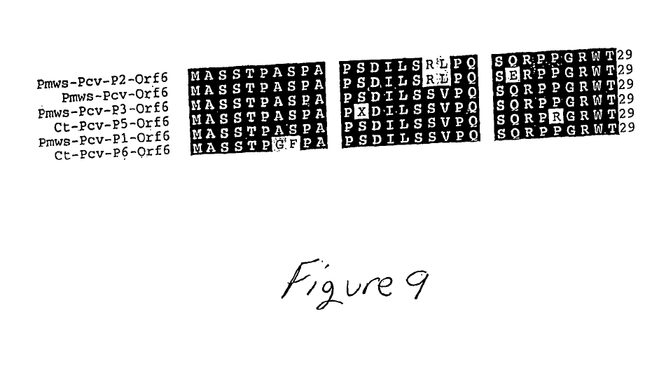

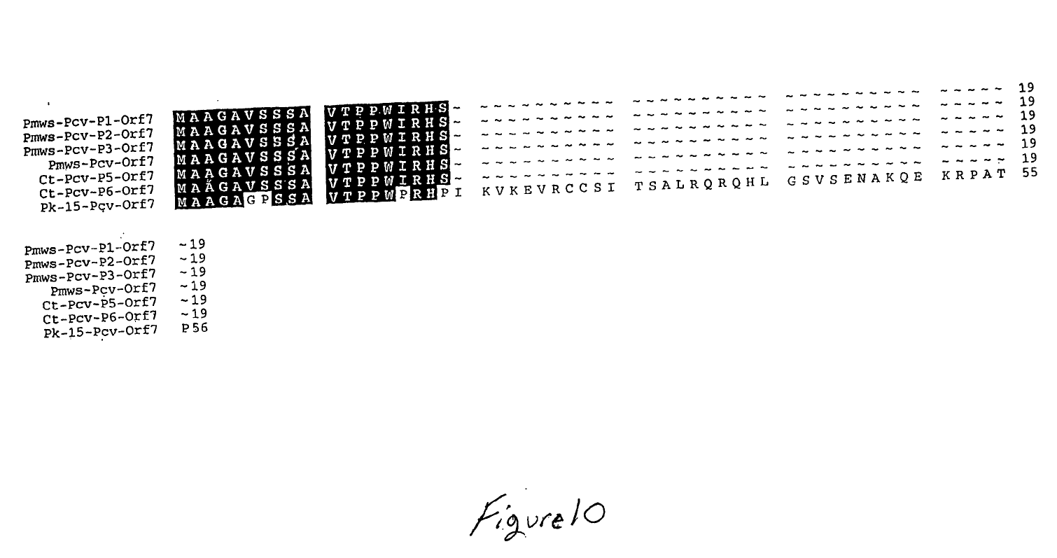

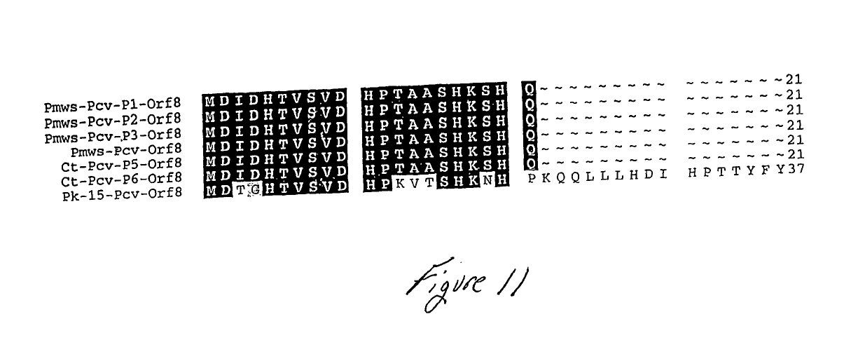

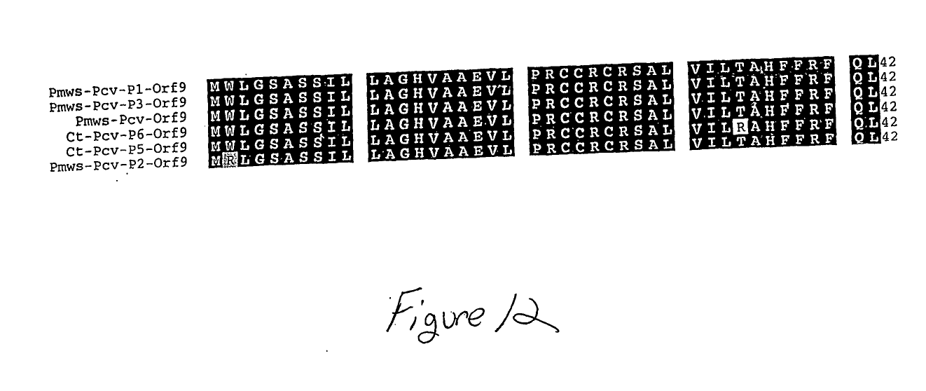

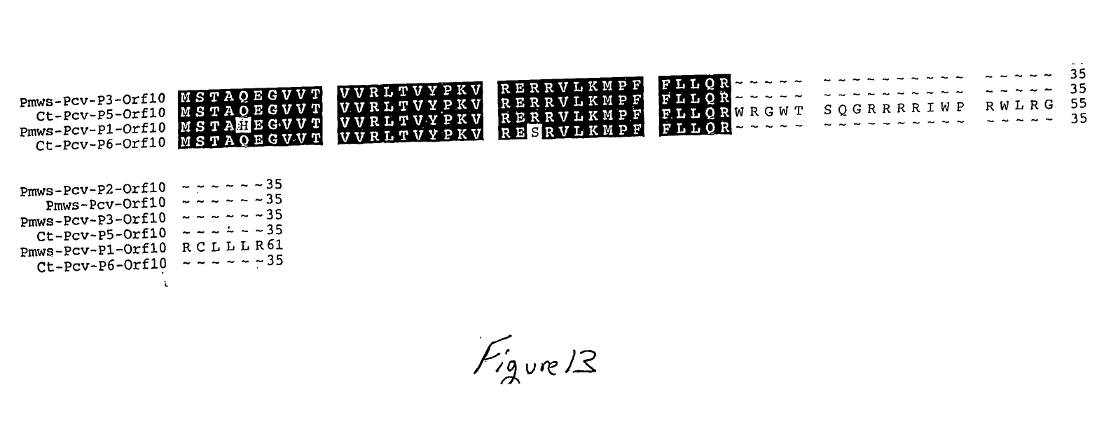

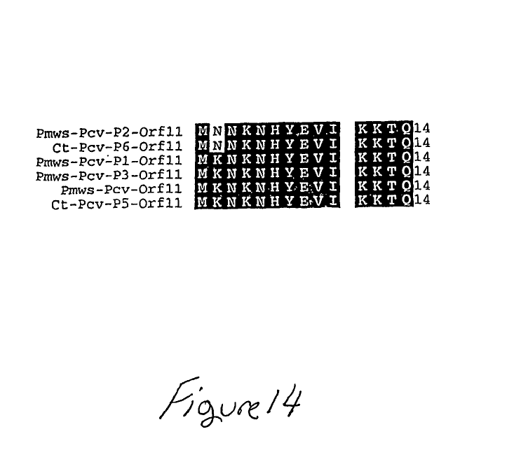

A “PCV polypeptide” (or “PCV protein”) refers to a polypeptide gene product encoded by a PCV open reading frame (ORF). Each PCV has 11 ORFs, and thus there is an ORF1, ORF2, ORF3, ORF4, ORF5, ORF6, ORF7, ORF8, ORF9, ORF10, and ORF11 for each strain, as set forth above. The characteristics of the ORFs and polypeptides they encode are set forth in the Tables in Example 1, infra, and in FIGS. 4-14.

The term “vaccine” refers to a composition protein or vector; the latter may also be loosely termed a “DNA vaccine”, although RNA vectors can be used as well) that can be used to elicit protective immunity in a recipient. It should be noted that to be effective, a vaccine of the invention can elicit immunity in a portion of the population, as some individuals may fail to mount a robust or protective immune response, or, in some cases, any immune response. This inability may stem from the individual's genetic background or because of an immunodeficiency condition (either acquired or congenital) or immunosuppression (e.g., treatment with immunosuppressive drugs to prevent organ rejection or suppress an autoimmune condition).

The term “immunotherapy” refers to a treatment regimen based on activation of a pathogen-specific immune response. A vaccine can be one form of immunotherapy. Charging dendritic cells with PCV polypeptide (a “PCV antigen”), optionally with a stimulatory cytokine such as GM-CSF or Flt3 ligand ex vivo (followed by transplantation into the subject) or in vivo is also a form of immunotherapy.

The term “protect” is used herein to mean prevent or treat, or both, as appropriate, an PCV infection in a subject. Thus, prophylactic administration of the vaccine can protect the recipient subject from PCV infection, e.g., to prevent infectious mononucleosis or lymphoproliferative diseases. Therapeutic administration of the vaccine or immunotherapy can protect the recipient from PCV-infection-mediated pathogenesis, e.g., to treat a disease or disorder such as PMWS or CT.

The term “subject” as used herein refers to an animal that supports PCV. In particular, the term refers to a pig.

The term “vector for expression in pigs” or “porcine expression vector” as used herein means that the vector at least includes a promoter that is effective in porcine cells, and preferably that the vector is safe and effective in pigs. Such a vector will, for example, omit extraneous genes not involved in developing immunity. If it is a viral vector, it will omit regions that permit replication and development of a robust infection, and will be engineered to avoid development of replication competence in vivo. Such vectors are preferably safe for use in pigs on a farm; in a more preferred embodiment, the vector is approved by a government regulatory agency (such as the United States Department of Agriculture (USDA)) for clinical testing or use in pigs. Specific vectors are described in greater detail below.

As used herein, the term “immunogenic polypeptide” means that the polypeptide is capable of eliciting a humoral or cellular immune response, and preferably both. An immunogenic polypeptide is also antigenic. A molecule is “antigenic” when it is capable of specifically interacting with an antigen recognition molecule of the immune system, such as an immunoglobulin (antibody) or T cell antigen receptor. An antigenic polypeptide contains an epitope of at least about 5, and preferably at least about 10, amino acids. An antigenic portion of a polypeptide, also called herein the epitope, can be that portion that is immunodominant for antibody or T cell receptor recognition, or it can be a portion used to generate an antibody to the molecule by conjugating the antigenic portion to a carrier polypeptide for immunization. A molecule that is antigenic need not be itself immunogenic, i.e., capable of eliciting an immune response without a carrier.

The term “adjuvant” refers to a compound or mixture that enhances the immune response to an antigen. An adjuvant can serve as a tissue depot that slowly releases the antigen and also as a lymphoid system activator that non-specifically enhances the immune response (Hood et al., Immunology, Second Ed., 1984, Benjamin/Cummings: Menlo Park, Calif., p. 384). Often, a primary challenge with an antigen alone, in the absence of an adjuvant, will fail to elicit a humoral or cellular immune response. Adjuvants include, but are not limited to, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, and potentially useful human adjuvants such as BCG (bacille Calmette-Guerin) and Corynebacterium parvum. Alternatively, or in addition, immunostimulatory proteins, as described below, can be provided as an adjuvant or to increase the immune response to a vaccine. Preferably, the adjuvant is pharmaceutically acceptable.

The phrase “pharmaceutically acceptable” or “veterinary acceptable” refers to molecular entities and compositions that are physiologically tolerable and do not typically produce an allergic or similar untoward reaction, such as gastric upset, dizziness and the like, when administered to an animal. Preferably, as used herein, the term “pharmaceutically acceptable” means approved by a regulatory agency of the Federal or a state government or listed in the U.S. Pharmacopeia or other generally recognized pharmacopeia for use in animals. The term “carrier” refers to a diluent, adjuvant, excipient, or vehicle with which the compound is administered. Sterile water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

As used herein, the term “isolated” means that the referenced material is removed from its native environment, e.g., a cell. Thus, an isolated biological material can be free of some or all cellular components, i.e., components of the cells in which the native material occurs naturally (e.g., cytoplasmic or membrane component). A material shall be deemed isolated if it is present in a cell extract or if it is present in a heterologous cell or cell extract. In the case of nucleic acid molecules, an isolated nucleic acid includes a PCR product, an isolated mRNA, a cDNA, or a restriction fragment. Isolated nucleic acid molecules include sequences inserted into plasmids, cosmids, artificial chromosomes, and the like, i.e., when it forms part of a chimeric recombinant nucleic aid construct. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated organelle, cell, or tissue is removed from the anatomical site in which it is found in an organism. An isolated material may be, but need not be, purified.

The term “purified” as used herein refers to material that has been isolated under conditions that reduce or eliminate the presence of unrelated materials, i.e., contaminants, including native materials from which the material is obtained. For example, a purified protein is preferably substantially free of other proteins or nucleic acids with which it is associated in a cell; a purified nucleic acid molecule is preferably substantially free of proteins or other unrelated nucleic acid molecules with which it can be found within a cell. As used herein, the term “substantially free” is used operationally, in the context of analytical testing of the material. Preferably, purified material substantially free of contaminants is at least 50% pure. Purity can be evaluated by chromatography, gel electrophoresis, immunoassay, composition analysis, biological assay, and other methods known in the art.

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

An “open-reading frame”, “coding sequence” or a sequence “encoding” an expression product, such as a RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein or enzyme. A coding sequence for a protein may include a start codon (usually ATG) and a stop codon.

A coding sequence is “under the control of” or “operatively associated with” transcriptional and translational control sequences in a cell when RNA polymerase transcribes the coding sequence into RNA, particularly mRNA, which is then trans-RNA spliced (if it contains introns) and translated into the protein encoded by the coding sequence.

The “expression control sequences” are transcriptional or translational control sequences including enhancer, repressor or promoter sequences.

A “promoter” or “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

The terms “vector”, “cloning vector” and “expression vector” mean the vehicle by which a DNA or RNA sequence (e.g. a foreign gene) can be introduced into a host cell, so as to transform the host and promote expression (e.g transcription and translation) of the introduced sequence. Vectors include plasmids, phages, viruses, etc.; they are discussed in greater detail below.

Vectors typically comprise the DNA of a transmissible agent, into which foreign DNA is inserted. A common way to insert one segment of DNA into another segment of DNA involves the use of enzymes called restriction enzymes that cleave DNA at specific sites (specific groups of nucleotides) called restriction sites.

A “cassette” refers to a DNA coding sequence or segment of DNA that codes for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct.” A common type of vector is a “plasmid”, which generally is a self-contained molecule of double-stranded DNA, usually of bacterial origin, that can readily accept additional (foreign) DNA and which can readily introduced into a suitable host cell. A plasmid vector often contains coding DNA and promoter DNA and has one or more restriction sites suitable for inserting foreign DNA. Coding DNA is a DNA sequence that encodes a particular amino acid sequence for a particular protein or enzyme. Promoter DNA is a DNA sequence which initiates, regulates, or otherwise mediates or controls the expression of the coding DNA. Promoter DNA and coding DNA may be from the same gene or from different genes, and may be from the same or different organisms. A large number of vectors, including plasmid and fungal vectors, have been described for replication and/or expression in a variety of eukaryotic and prokaryotic hosts. Non-limiting examples include pKK plasmids (Clonetech), pUC plasmids, pET plasmids (Novagen, Inc., Madison, Wis.), pRSET or pREP plasmids (Invitrogen, San Diego, Calif.), or pMAL plasmids (New England Biolabs, Beverly, Mass.), and many appropriate host cells, using methods disclosed or cited herein or otherwise known to those skilled in the relevant art. Recombinant cloning vectors will often include one or more replication systems for cloning or expression, one or more markers for selection in the host, e.g antibiotic resistance, and one or more expression cassettes.

The terms “express” and “expression” mean allowing or causing the information in a gene or DNA sequence to become manifest, for example producing a protein by activating the cellular functions involved in transcription and translation of a corresponding gene or DNA sequence. A DNA sequence is expressed in or by a cell to form an “expression product” such as a protein. The expression product itself, e.g the resulting protein, may also be said to be “expressed” by the cell. An expression product can be characterized as intracellular, extracellular or secreted. The term “intracellular” means something that is inside a cell. The term “extracellular” means something that is outside a cell. A substance is “secreted” by a cell if it appears in significant measure outside the cell, from somewhere on or inside the cell.

The term “transfection” means the introduction of a foreign nucleic acid into a cell. The term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. The introduced gene or sequence may also be called a “cloned” or “foreign” gene or sequence, may include regulatory or control sequences, such as start, stop, promoter, signal, secretion, or other sequences used by a cell's genetic machinery. The gene or sequence may include nonfunctional sequences or sequences with no known function. A host cell that receives and expresses introduced DNA or RNA has been “transformed” and is a “transformant” or a “clone.” The DNA or RNA introduced to a host cell can come from any source, including cells of the same genus or species as the host cell, or cells of a different genus or species.

The term “host cell” means any cell of any organism that is selected, modified, transformed, grown, or used or manipulated in any way, for the production of a substance by the cell, for example the expression by the cell of a gene, a DNA or RNA sequence, a protein or an enzyme. Suitable host cells include primary macrophages, particularly porcine macrophages, or such a macrophage cell line, porcine kidney cells, or other mammalian cells in which PCV can produce virus, or that support a viral infection, or both.

The term “expression system” means a host cell and compatible vector under suitable conditions, e.g. for the expression of a protein coded for by foreign DNA carried by the vector and introduced to the host cell. Common expression systems include E. coli host cells and plasmid vectors, insect host cells and Baculovirus vectors, and mammalian host cells and vectors. In a specific embodiment, the protein of interest is expressed in COS-1 or C₂C₁₂ cells. Other suitable cells include CHO cells, HeLa cells, 293T (human kidney cells), mouse primary myoblasts, and NIH 3T3 cells.

“Sequence-conservative variants” of a polynucleotide sequence are those in which a change of one or more nucleotides in a given codon position results in no alteration in the amino acid encoded at that position.

“Function-conservative variants” are those in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids with similar properties are well known in the art. For example, arginine, histidine and lysine are hydrophilic-basic amino acids and may be interchangeable. Similarly, isoleucine, a hydrophobic amino acid, may be replaced with leucine, methionine or valine. Such changes are expected to have little or no effect on the apparent molecular weight or isoelectric point of the protein or polypeptide. Amino acids other than those indicated as conserved may differ in a protein or enzyme so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” also includes a polypeptide or enzyme which has at least 60% amino acid identity as determined by BLAST or FASTA algorithms, preferably at least 75%, most preferably at least 85%, and even more preferably at least 90%, and which has the same or substantially similar properties or functions as the native or parent protein or enzyme to which it is compared.

As used herein, the term “homologous” in all its grammatical forms and spelling variations refers to the relationship between proteins that possess a “common evolutionary origin,” including proteins from superfamilies (e.g., the immunoglobulin superfamily) and homologous proteins from different species (e.g., myosin light chain, etc.) (Reeck et al., Cell 50:667, 1987). Such proteins (and their encoding genes) have sequence homology, as reflected by their sequence similarity, whether in terms of percent similarity or the presence of specific residues or motifs at conserved positions.

Accordingly, the term “sequence similarity” in all its grammatical forms refers to the degree of identity or correspondence between nucleic acid or amino acid sequences of proteins that may or may not share a common evolutionary origin (see Reeck et al., supra). However, in common usage and in the instant application, the term “homologous,” when modified with an adverb such as “highly,” may refer to sequence similarity and may or may not relate to a common evolutionary origin.

In a specific embodiment, two DNA sequences are “substantially homologous” or “substantially similar” when at least about 80%, and most preferably at least about 90 or 95%) of the nucleotides match over the defined length of the DNA sequences, as determined by sequence comparison algorithms, such as BLAST, FASTA, DNA Strider, etc. An example of such a sequence is an allelic or species variant of the specific genes of the invention. Sequences that are substantially homologous can be identified by comparing the sequences using standard software available in sequence data banks, or in a Southern hybridization experiment under, for example, stringent conditions as defined for that particular system.

Similarly, in a particular embodiment, two amino acid sequences are “substantially homologous” or “substantially similar” when greater than 80% of the amino acids are identical, or greater than about 90% are similar (functionally identical). Preferably, the similar or homologous sequences are identified by alignment using, for example, the GCG (Genetics Computer Group, Program Manual for the GCG Package, Version 7, Madison, Wis.) pileup program, or any of the programs described above (BLAST, FASTA, etc.).

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., supra). The conditions of temperature and ionic strength determine the “stringency” of the hybridization. For preliminary screening for homologous nucleic acids, low stringency hybridization conditions, corresponding to a Tₘ (melting temperature) of 55° C., can be used, e.g., 5×SSC, 0.1% SDS, 0.25% milk, and no formamide; or 30% formamide, 5×SSC, 0.5% SDS). Moderate stringency hybridization conditions correspond to a higher Tₘ, e.g., 40% formamide, with 5× or 6×SCC. High stringency hybridization conditions correspond to the highest Tₘ, e.g., 50% formamide, 5× or 6×SCC. SCC is a 0.15 M NaCl, 0.015M Na-citrate. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ have been derived (see Sambrook et al., supra, 9.50-9.51). For hybridization with shorter nucleic acids, i.e., oligonucleotides, the position of mismatches becomes more important, and the length of the oligonucleotide determines its specificity (see Sambrook et al., supra, 11.7-11.8). A minimum length for a hybridizable nucleic acid is at least about 10 nucleotides; preferably at least about 15 nucleotides; and more preferably the length is at least about 20 nucleotides.

In a specific embodiment, the term “standard hybridization conditions” refers to a Tₘ of 55° C., and utilizes conditions as set forth above. In a preferred embodiment, the Tₘ is 60° C.; in a more preferred embodiment, the Tₘ is 65° C. In a specific embodiment, “high stringency” refers to hybridization and/or washing conditions at 68° C. in 0.2×SSC, at 42° C. in 50% formamide, 4×SSC, or under conditions that afford levels of hybridization equivalent to those observed under either of these two conditions.

As used herein, the term “oligonucleotide” refers to a nucleic acid, generally of at least 10, preferably at least 15, and more preferably at least 20 nucleotides, preferably no more than 100 nucleotides, that is hybridizable to a genomic DNA molecule, a cDNA molecule, or an mRNA molecule encoding a gene, mRNA, cDNA, or other nucleic acid of interest. Oligonucleotides can be labeled, e.g., with ³²P-nucleotides or nucleotides to which a label, such as biotin, has been covalently conjugated. In one embodiment, a labeled oligonucleotide can be used as a probe to detect the presence of a nucleic acid. In another embodiment, oligonucleotides (one or both of which may be labeled) can be used as PCR primers, either for cloning full length or a fragment of the gene, or to detect the presence of nucleic acids encoding the protein. In a further embodiment, an oligonucleotide of the invention can form a triple helix with a DNA molecule. Generally, oligonucleotides are prepared synthetically, preferably on a nucleic acid synthesizer. Accordingly, oligonucleotides can be prepared with non-naturally occurring phosphoester analog bonds, such as thioester bonds, etc.

### Cloning and Expression of PCV

The inventors have succeeded in cloning and sequencing seven porcine circovirus (PCV) strains: one of a virus strain isolated in the late 1960's (herein named CT-PCV 7) and originated from neonatal pigs with congenital tremors (CT) of Type A2; two new PCV isolates from pigs showing CT (herein named CT-PCV-P5, and CT-PCV-P6); and four PCV strains obtained from pigs showing signs of postweaning multisystemic wasting syndrome (PMWS) (herein named PMWS-PCV-P 1, PMWS-PCV-P2, PMWS-PCV-P3, and PMWS-PCV-P4).

It was discovered that the PMWS-PCV isolates yielded an approximately 99% nucleotide sequence identity with each other. Furthermore although the new CT-PCV isolates from the late 1990s and the old CT-PCV isolate from the late 1960's originated from neonatal pigs with CT type A2, they shared only 72% nucleotide sequence identity. The genomes of the 2 new CT-PCVs share high sequence homology with the type 2 PMWS-PCV isolates. On the other hand the CT-PCV-7 strain was found to be very close to type 1 PK-15-PCV variants.

A subject of the present invention is, thus, an isolated nucleic acid from PCV, which nucleic acid comprises a sequence selected from the group consisting of SEQ ID NO. 1 to SEQ ID NO. 7

Another subject of the invention is an isolated nucleic acid from PCV which has a sequence identical to a sequence selected from the group consisting of SEQ ID NO. 1 to SEQ ID NO. 7.

The nucleic acid sequences of the invention may be useful to design probes or primers for detecting the presence of a PCV nucleic acid in a biological sample. Such probes or primer may be more particularly in the form of oligonucleotides, that specifically hybridize to PCV nucleic acid sequences under conditions of high stringency. Such oligonucleotides, which preferably comprise at least about 20 bases that has a sequence found in 20 contiguous bases of SEQ ID NO. 1 to SEQ ID NO. 7 or a complement thereof.

Eleven open-reading frame (ORF) sequences have been determined and are presented in Tables 2 and 4 of the Examples. The corresponding polypeptide sequences are also shown on FIGS. 4 to 14.

The invention, thus, also provides a nucleic acid from PCV, which nucleic acid comprises a sequence coding for a circovirus polypeptide having a sequence selected from the group consisting of the aminoacid sequences coded by any of ORF1 to ORF 11 of any of the sequences of SEQ ID NO. 1 to SEQ ID NO. 7. More particularly the nucleic acid of the invention comprises a sequence selected from any of ORF1 to ORF 11 of any of the sequences of SEQ ID NO. 1 to SEQ ID NO. 7. Among these, ORFs 1, 2, 3 or 4, are particularly interesting.

The present invention encompasses conservative sequences, that is to say the sequences which do not change the functionality or the strain-specificity of the sequence described or of the polypeptides encoded by this sequence. These sequences are also called “function-conservative variants”. The sequences differing by degeneracy of the code, which are called “sequence-conservative variants”, also are encompassed.

The invention also covers the equivalent sequences in the sense that they are capable of hybridizing with the above sequence under high stringency conditions and/or have a very high homology with the strains of the invention.

Cloning vectors comprising any of these nucleic acid sequences are also part of the invention. The preparation of such vectors is well-known by one skilled in the art and is described in the above definitions.

These nucleic acid sequences and their fragments can be advantageously used for in vitro or in vivo expression of a polypeptide with the aid of appropriate expression vectors.

These vectors more particularly comprise a sequence selected from the group consisting of any of ORF1 to ORF11 of any of the sequences of SEQ ID NO. 1 to SEQ ID NO. 7., operatively associated with an expression control sequence.

The vectors of the invention may be used to transfect host cells, which are also part of the present invention. Vectors are introduced into the desired host cells by methods known in the art, e.g., transfection, electroporation, microinjection, transduction, cell fusion, DEAE dextran, calcium phosphate precipitation, lipofection (lysome fusion), use of a gene gun, oraDNA vector transporter (see, e.g., Wu et al., J. Biol. Chem. 267:963-967, 1992; Wu and Wu, J. Biol. Chem. 263:14621-14624, 1988; Hartmutetal., Canadian Patent Application No. 2,012,311, filed Mar. 15, 1990).

The invention further provides a method for producing a PCV protein, which method comprises culturing a cell transfected with an expression vector as above-defined under conditions that result in expression of the nucleic acid coding for a circovirus protein. E. coli or baculovirus are the expression systems that may be used (U.S. Pat. No. 4,745,051) for that purpose. The coding sequences may be integrated into the baculovirus genome (e.g the baculovirus Autographa californica Nuclear Polyhedrosis Virus AcNPV) and the latter can be then propagated on insect cells, e.g. Spodoptera frugiperda Sf9 (deposit ATCC CRL 1711).

More generally the invention is directed to the expression of PCV polypeptides or proteins in vitro, in vivo or ex vivo. For these various purposes, one skilled in the art may select any suitable expression system, as detailed below.

### Expression Systems

A wide variety of host/expression vector combinations (i.e., expression systems) may be employed in expressing the polypeptides of this invention. Useful expression vectors, for example, may consist of segments of chromosomal, non-chromosomal and synthetic DNA sequences. Suitable vectors include derivatives of SV40 and known bacterial plasmids, e.g., E. coli plasmids col E1, pCR1, pBR322, pMal-C2, pET, pGEX (Smith et al., Gene 67:31-40, 1988), pMB9 and their derivatives, plasmids such as RP4; gram positive vectors such as Strep. gardonii; phage DNAS, e.g., the numerous derivatives of phage 1 μl, e.g., NM989, and other phage DNA, e.g., M13 and filamentous single stranded phage DNA; yeast plasmids such as the 2μ plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

Expression of the protein or polypeptide may be controlled by any promoter/enhancer element known in the art, but these regulatory elements must be functional in the host selected for expression. Promoters which may be used to control gene expression include, but are not limited to, cytomegalovirus (CMV) promoter, the SV40 early promoter region (Benoist and Chambon, 1981, Nature 290:304-310), the promoter contained in the 3′ long terminal repeat of Rous sarcoma virus (Yamamoto, et al., Cell 22:787-797, 1980), the herpes thymidine kinase promoter (Wagner et al., Proc. Natl. Acad. Sci. U.S.A. 78:1441-1445, 1981), the regulatory sequences of the metallothionein gene (Brinster et al., Nature 296:39-42, 1982); prokaryotic expression vectors such as the b-lactamase promoter (Villa-Komaroff, et al., Proc. Natl. Acad. Sci. U.S.A. 75:3727-3731, 1978), or the tac promoter (DeBoer, et al., Proc. Natl. Acad. Sci. U.S.A. 80:21-25,1983); see also “Useful proteins from recombinantbacteria” in Scientific American, 242:74-94,1980; promoter elements fromyeast or other fungi such as the Gal 4 promoter, the ADC (alcohol dehydrogenase) promoter, PGK (phosphoglycerol kinase) promoter, alkaline phosphatase promoter; and control regions that exhibit hematopoietic tissue specificity, in particular: beta-globin gene control region which is active in myeloid cells (Mogram et al., Nature 315:338-340, 1985; Kollias et al., Cell 46:89-94, 1986), hematopoietic stem cell differentiation factor promoters, erythropoietin receptor promoter (Maouche et al., Blood, 15:2557, 1991), etc; and control regions that exhibit mucosal epithelial cell specificity.

Preferred vectors, particularly for cellular assays in vitro and vaccination in vivo or ex vivo, are viral vectors, such as lentiviruses, retroviruses, herpes viruses, adenoviruses, adeno-associated viruses, vaccinia viruses, baculoviruses, and other recombinant viruses with desirable cellular tropism. Thus, a vector encoding an immunogenic polypeptide can be introduced in vivo, ex vivo, or in vitro using a viral vector or through direct introduction of DNA. Expression in targeted tissues can be effected by targeting the transgenic vector to specific cells, such as with a viral vector or a receptor ligand, or by using a tissue-specific promoter, or both. Targeted gene delivery is described in International Patent Publication WO 95/28494, published October 1995.

Viral vectors commonly used for in vivo or ex vivo targeting and vaccination procedures are DNA-based vectors and retroviral vectors. Methods for constructing and using viral vectors are known in the art (see, e.g., Miller and Rosman, BioTechniques, 7:980-990, 1992). Preferably, the viral vectors are replication defective, that is, they are unable to replicate autonomously in the target cell. Preferably, the replication defective virus is a minimal virus, i.e., it retains only the sequences of its genome which are necessary for encapsidating the genome to produce viral particles.

DNA viral vectors include an attenuated or defective DNA virus, such as but not limited to herpes simplex virus (HSV), papillomavirus, Epstein Barr virus (EBV), adenovirus, adeno-associated virus (AAV), vaccinia virus, and the like. Examples of particular vectors include, but are not limited to, a defective herpes virus 1 (HSV1) vector (Kaplitt et al., Molec. Cell. Neurosci. 2:320-330, 1991; International Patent Publication No. WO 94/21807, published Sep. 29, 1994; International Patent Publication No. WO 92/05263, published Apr. 2, 1994); an attenuated adenovirus vector, such as the vector described by Stratford-Perricaudet et al. (J. Clin. Invest. 90:626-630, 1992; see also La Salle et al., Science 259:988-990, 1993); and a defective adeno-associated virus vector (Samulski et al., J. Virol. 61:3096-3101, 1987; Samulski et al., J. Virol. 63:3822-3828,1989; Lebkowski et al., Mol. Cell. Biol. 8:3988-3996, 1988).

Various companies produce viral vectors commercially, including but by no means limited to Avigen, Inc. (Alameda, Calif.; AAV vectors), Cell Genesys (Foster City, Calif.; retroviral, adenoviral, AAV vectors, and lentiviral vectors), Clontech (retroviral and baculoviral vectors), Genovo, Inc. (Sharon Hill, Pa.; adenoviral and AAV vectors), Genvec (adenoviral vectors), IntroGene (Leiden, Netherlands; adenoviral vectors), Molecular Medicine (retroviral, adenoviral, AAV, and herpes viral vectors), Norgen (adenoviral vectors), Oxford BioMedica (Oxford, United Kingdom; lentiviral vectors), and Transgene (Strasbourg, France; adenoviral, vaccinia, retroviral, and lentiviral vectors).

Adenovirus vectors. Adenoviruses are eukaryotic DNA viruses that can be modified to efficiently deliver a nucleic acid of the invention to a variety of cell types. Various serotypes of adenovirus exist. Of these serotypes, preference is given, within the scope of the present invention, to using type 2 or type 5 human adenoviruses (Ad 2 or Ad 5) or adenoviruses of animal origin (see WO94/26914). Those adenoviruses of animal origin which can be used within the scope of the present invention include adenoviruses of canine, bovine, murine (example: Mavl, Beard et al., Virology 75 (1990) 81), ovine, porcine, avian, and simian (example: SAV) origin. Preferably, the adenovirus of animal origin is a canine adenovirus, more preferably a CAV2 adenovirus (e.g. Manhattan or A26/61 strain (ATCC VR-800), for example). Various replication defective adenovirus and minimum adenovirus vectors have been described (WO94/26914, WO95/02697, WO94/28938, WO94/28152, WO94/12649, WO95/02697 WO96/22378). The - replication defective recombinant adenoviruses according to the invention can be prepared by any technique known to the person skilled in the art (Levrero et al., Gene 101:195 1991; EP 185 573; Graham, EMBO J. 3:2917, 1984; Graham et al., J. Gen. Virol. 36:59, 1977). Recombinant adenoviruses are recovered and purified using standard molecular biological techniques, which are well known to one of ordinary skill in the art.

Adeno-associated viruses. The adeno-associated viruses (AAV) are DNA viruses of relatively small size which can integrate, in a stable and site-specific manner, into the genome of the cells which they infect. They are able to infect a wide spectrum of cells without inducing any effects on cellular growth, morphology or differentiation, and they do not appear to be involved in human pathologies. The AAV genome has been cloned, sequenced and characterized. The use of vectors derived from the AAVs for transferring genes in vitro and in vivo has been described (see WO 91/18088; WO 93/09239; U.S. Pat. No. 4,797,368, U.S. Pat. No. 5,139,941, EP 488 528). The replication defective recombinant AAVs according to the invention can be prepared by cotransfecting a plasmid containing the nucleic acid sequence of interest flanked by two AAV inverted terminal repeat (ITR) regions, and a plasmid carrying the AAV encapsidation genes (rep and cap genes), into a cell line which is infected with a human helper virus (for example an adenovirus). The AAV recombinants which are produced are then purified by standard techniques.

Retrovirus vectors. In another embodiment the gene can be introduced in a retroviral vector, e.g., as described in Anderson et al., U.S. Pat. No. 5,399,346; Mann et al., Cell 33:153 1983, Temin et al, U.S. Pat. No. 4,650,764; Temin et al., U.S. Pat. No. 4,980,289; Markowitz et al., J. Virol. 62:1120 1988, Temin et al., U.S. Pat. No. 5,124,263; EP 453242, EP178220; Bernstein et al. Genet. Eng. 7 (1985) 235; McCormick, BioTechnology 3 (1985) 689; International Patent Publication No. WO 95/07358, published Mar. 16, 1995, by Dougherty et al.; and Kuo et aL, Blood 82:845, 1993,. The retroviruses are integrating viruses which infect dividing cells. The retrovirus genome includes two LTRs, an encapsidation sequence and three coding regions (gag, pol and env). In recombinant retroviral vectors, the gag, pol and env genes are generally deleted, in whole or in part, and replaced with a heterologous nucleic acid sequence of interest. These vectors can be constructed from different types of retrovirus, such as, HIV, MoMuLV (“murine Moloney leukaemia virus” MSV (“murine Moloney sarcoma virus”), HaSV (“Harvey sarcoma virus”); SNV (“spleen necrosis virus”); RSV (“Rous sarcoma virus”) and Friend virus. Suitable packaging cell lines have been described in the prior art, in particular the cell line PA317 (U.S. Pat. No. 4,861,719); the PsiCRIP cell line (WO 90/02806) and the GP+envAm-12 cell line (WO 89/07150). In addition, the recombinant retroviral vectors can contain modifications within the LTRs for suppressing transcriptional activity as well as extensive encapsidation sequences which may include a part of the gag gene (Bender et al., J. Virol. 61:1639, 1987). Recombinant retroviral vectors are purified by standard techniques known to those having ordinary skill in the art.

Retrovirus vectors can also be introduced by DNA viruses, which permits one cycle of retroviral replication and amplifies tranfection efficiency (see WO 95/22617, WO 95/26411, WO 96/39036, WO 97/19182).

Lentivirus vectors. In another embodiment, lentiviral vectors can be used as agents for the direct delivery and sustained expression of a transgene in several tissue types, including brain, retina, muscle, liver and blood. The vectors can efficiently transduce dividing and nondividing cells in these tissues, and maintain long-term expression of the gene of interest. For a review, see, Naldini, Curr. Opin. Biotechnol., 9:457-63, 1998; see also Zufferey, et al., J. Virol., 72:9873-80, 1998). Lentiviral packaging cell lines are available and known generally in the art. They facilitate the production of high-titer lentivirus vectors for gene therapy. An example is a tetracycline-inducible VSV-G pseudotyped lentivirus packaging cell line which can generate virusparticles at titers greater than 106 IU/ml for at least 3 to 4 days (Kafri, et al., J. Virol., 73: 576-584, 1999). The vector produced by the inducible cell line can be concentrated as needed for efficiently transducing nondividing cells in vitro and in vivo.

Non-viral vectors. In another embodiment, the vector can be introduced in vivo by lipofection, as naked DNA, or with other transfection facilitating agents (peptides, polymers, etc.). Synthetic cationic lipids can be used to prepare liposomes for in vivo transfection of a gene encoding a marker (Felgner, et. al., Proc. Natl. Acad. Sci. U.S.A. 84:7413-7417, 1987; Felgner and Ringold, Science 337:387-388, 1989; see Mackey, et al., Proc. Natl. Acad. Sci. U.S.A. 85:8027-8031, 1988; Ulmer et al., Science 259:1745-1748, 1993). Useful lipid compounds and compositions for transfer of nucleic acids are described in International Patent Publications WO95/18863 and WO96/17823, and in U.S. Pat. No. 5,459,127. Lipids may be chemically coupled to other molecules for the purpose of targeting (see Mackey, et al., supra). Targeted peptides, e.g., hormones or neurotransmitters, and proteins such as antibodies, or non-peptide molecules could be coupled to liposomes chemically.

Other molecules are also useful for facilitating transfection of a nucleic acid in vivo, such as a cationic oligopeptide (e.g., International Patent Publication WO95/21931), peptides derived from DNA binding proteins (e.g., International Patent Publication WO96/25508), or a cationic polymer (e.g., International Patent Publication WO95/21931).

It is also possible to introduce the vector in vivo as a naked DNA plasmid. Naked DNA vectors for gene therapy can be introduced into the desired host cells by methods known in the art, e.g., electroporation, microinjection, cell fusion, DEAE dextran, calcium phosphate precipitation, use of a gene gun (ballistic transfection), or use of a DNA vector transporter (see, e.g., Wu et al., J. Biol. Chem. 267:963-967, 1992; Wu and Wu, J. Biol. Chem. 263:14621-14624, 1988; Hartmut et al., Canadian Patent Application No.2,012,311, filed Mar. 15, 1990; Williams et al., Proc. Natl. Acad. Sci. USA 88:2726-2730, 1991). Receptor-mediated DNA delivery approaches can also be used (Curiel et al., Hum. Gene Ther. 3:147-154, 1992; Wu and Wu, J. Biol. Chem. 262:4429-4432, 1987). U.S. Pat. Nos. 5,580,859 and 5,589,466 disclose delivery of exogenous DNA sequences, free of transfection facilitating agents, in a mammal. Recently, a relatively low voltage, high efficiency in vivo DNA transfer technique, termed electrotransfer, has been described (Mir et al., C. P. Acad. Sci., 321:893, 1998; WO 99/01157; WO 99/01158; WO 99/01175).

### Purification of the PCV polypeptides

The polypeptide that is so produced may be recovered and preferably purified. Methods for purification are well-known in the art. The purification methods including, without limitation, preparative disc-gel electrophoresis and isoelectric focusing; affinity, HPLC, reversed-phase HPLC, gel filtration or size exclusion, ion exchange and partition chromatography; precipitation and salting-out chromatography; extraction; and countercurrent distribution. For some purposes, it is preferable to produce the polypeptide in a recombinant system in which the protein contains an additional sequence tag that facilitates purification, such as, but not limited to, a polyhistidine sequence, or a sequence that specifically binds to an antibody, such as FLAG and GST. The polypeptide can then be purified from a crude lysate of the host cell by chromatography on an appropriate solid-phase matrix. Alternatively, antibodies produced against the protein or against peptides derived therefrom can be used as purification reagents.

### Anti-PCV Antibodies

Such antibodies include but are not limited to polyclonal, monoclonal, chimeric, single chain, Fab fragments, and an Fab expression library.

Various procedures known in the art may be used for the production of polyclonal antibodies to PCV polypeptides or derivative or analog thereof. For the production of antibody, various host animals can be immunized by injection with the antigenic polypeptide, including but not limited to rabbits, mice, rats, sheep, goats, etc. Preferably, the immunized animal is of the same species as the animal who will receive the antibodies in passive immunization, to avoid allergic reactions to the antibodies.

For preparation of monoclonal antibodies directed toward the PCV polypeptides, any technique that provides for the production of antibody molecules by continuous cell lines in culture may be used. These include but are not limited to the hybridoma technique originally developed by Kohler and Milstein (Nature 256:495-497, 1975), as well as the trioma technique, the human B-cell hybridoma technique (Kozbor et al., Immunology Today 4:72, 1983; Cote et al., Proc. Natl. Acad. Sci. U.S.A. 80:2026-2030, 1983), and the EBV-hybridoma technique to produce human monoclonal antibodies (Cole et al., in Monoclonal Antibodies and Cancer Therapy, Alan R. Liss, Inc., pp. 77-96, 1985). In an additional embodiment of the invention, monoclonal antibodies can be produced in germ-free animals (International Patent Publication No. WO 89/12690, published 28 December, 1989).

According to the invention, techniques described for the production of single chain antibodies (U.S. Pat. Nos. 5,476,786 and 5,132,405 to Huston; U.S. Pat. No. 4,946,778) can be adapted to produce the PCV polypeptide-specific single chain antibodies. Indeed, these genes can be delivered for expression in vivo. An additional embodiment of the invention utilizes the techniques described for the construction of Fab expression libraries (Huse et al., Science 246:1275-1281, 1989) to allow rapid and easy identification of monoclonal Fab fragments with the desired specificity for a PCV polypeptide, or its derivatives, or analogs.

Antibody fragments which contain the idiotype of the antibody molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment, and the Fab fragments which can be generated by treating the antibody molecule with papain and a reducing agent.

In the production of antibodies, screening for the desired antibody can be accomplished by techniques known in the art, e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention.

### Culturing PCV In Vitro

The present invention further relates to a method of culturing a porcine circovirus strain which method comprises introducing a nucleic acid comprising a sequence selected from the group consisting of SEQ ID NO:1 TO SEQ ID NO:6 into a suitable host cell under conditions that result in the production of porcine circovirus particles having a genome that comprises a sequence selected from the group consisting of SEQ ID NO:1 TO SEQ ID NO:6.

Uninfected cells of any type, preferably pig cells (e.g, cells derived from Sus scrofa or Tayassu tajacu), more preferably neuronally derived pig cells (e.g, glial cells), pig kidney cells (e.g., PK-15 cells) or pig macrophage cells may be infected by introduction of PCV nucleic acids of the invention (e.g., single or preferably double stranded genomic DNA or one or more plasmids comprising PCV genomic DNA) into the cells. Infection or transfection of a host cell line is a technique which is commonly known in the art and may be performed by any practitioner of ordinary skill in the art. For example, Example 1 includes a procedure for transfecting PK-15 cells with PCV DNA (see Transfection of cloned PCV DNAs and detection of PCV).

Once infected, one or more clones from the infected cell line may be selected and propagated. The cells from the selected clones may be stored (e.g, frozen) and used as a master cell bank from which samples may be taken and used to generate multiple working cell lines. Viral particles which are used for vaccine production and as a source of viral proteins may be obtained from the working cell lines. Specifically, a working cell line may be produced by thawing a sample of a frozen master cell line and expanding the cells in culture; the cells in the expanded cell culture are used as the working cell line. For example, the master cell line may be thawed and grown in a Nunc Cell Factory (Nalge Nunc International; Rochester, N.Y.) for production of a large quantity of cells.

Viral particles may be obtained from the working cell lines by methods which are commonly known to those of ordinary skill in the art. For example, viral particles in the culture supernatant of the working cell lines may be harvested, filtered, purified (e.g, by gradient centrifugation) and used for vaccine generation.

### Diagnosis of Congenital Tremors

The evidence of an association between porcine circovirus and congenital tremors (CT) allows the inventors to present a method for diagnosing a pathological cause of congenital tremors (CT) in a pig or its progeny, which method comprises determining whether the pig has been infected by a porcine circovirus.

As used herein, the term “diagnosis” refers to the identification of the disease at any stage of its development, and also includes the determination of a predisposition of a foetus or new-born piglet to develop the disease or a predisposition of a sow to transmit the disease to the foetus.

The diagnostic method of the invention may involve the detection of any PCV strain of Type 1 or Type 2. The repartition of PCV strains between the two types is shown in FIG. 3. PCV strains of Type 1 more particularly include PK-15 PCV or the so-called “CT-PCV-7” strain that comprises nucleic acid sequence SEQ ID NO. 7. PCV strains of Type 2 more particularly include the strains that comprise a nucleic acid sequence of any of SEQ ID NO. 1 to SEQ ID NO. 6. PCV strains that are associated with PMWS (“PMWS-PCV”) are also included in the target PCV strains.

The diagnostic method of the invention may be performed by any standard technique well-known by one skilled in the art, as reviewed for example, in the International Application WO 99/18214.

In a first embodiment, the determination of the infection may encompass detecting the presence of a PCV nucleic acid in a biological sample of the test pig.

In a second embodiment, the determination of the infection may encompass detecting the presence of a PCV polypeptide in a biological sample of the test pig

In a third embodiment, the determination of the infection may be effected by detecting the presence of an antibody directed against a PCV polypeptide in a biological sample of the test pig.

The biological sample may be of any kind, including a fluid sample (blood, plasma, serum, cerebrospinal fluid, etc.) or an organ or tissue sample (ganglions, liver, etc.). Cells or cell extracts from the central nervous system may be used more particularly for post-mortem diagnosis.

However preferred test samples and methods are those that can be easily implemented by a veterinarian or the animal breeder on the farm. Accordingly, Western blot, immunofluorescence, ELISA or immunochromatography suit these applications very well.

In ELISA assays, a polypeptide of the invention or epitopic fragment thereof are immobilized onto a selected surface, for example, a surface capable of binding proteins such as the wells of a polystyrene microtiter plate. After washing to remove incompletely adsorbed polypeptides, a nonspecific protein such as a solution of bovine serum albumin (BSA) that is known to be antigenically neutral with regard to the test sample may be bound to the selected surface. This allows for blocking of nonspecific adsorption sites on the immobilizing surface and thus reduces the background caused by nonspecific bindings of antisera onto the surface. The immobilizing surface is then contacted with a sample, such as clinical or biological materials, to be tested in a manner conductive to immune complex (antigen/antibody) formation. This may include diluting the sample with diluents, such as solutions of BSA, bovine gamma globulin (BGG) and/or phosphate buffered saline (PBS)/Tween. The sample is then allowed to incubate for from 2 to 4 hours, at temperatures such as of the order of about 25° to 37° C. Following incubation, the sample-contacted surface is washed to remove non-immunocomplexed material. The washing procedure may include washing wiht a solution, such as PBS/Tween or borate buffer. Following formation of specific immunocomplexes between the test sample and the bound polypeptide, and subsequent washing, the occurrence, and an even amount of immunocomplex formation may be determined by subjecting the immunocomplex to a second antibody having specificity for the first antibody. To provide detecting means, the second antibody may have an associated activity such as an enzymatic activity that will generate, for example, a color development upon incubating with an appropriate chromogenic substrate. Quantification may then be achieved by measuring the degree of color generation using, for example, a visible spectra spectrophotometer.

As to immunochromatography techniques, one skilled in the art may find detailed information in F. Zurk et al., Clin. Chem. 31/7, 1144-1150 (1985), as well as in patents or patent applications WO-88/08534; WO-91/12528; EP 291 176; EP 299 428; EP 291 194; EP 284 232; U.S. Pat. No. 5,120,643; U.S. Pat. No. 5,030,558; U.S. Pat. No. 5,266,497; U.S. Pat. No. 4,740,468; U.S. Pat. No. 5,266,497; U.S. Pat. No. 4,855,240; U.S. Pat. No. 5,451,504; U.S. Pat. No. 5,141,850; U.S. Pat. No. 5,232,835; U.S. Pat. No. 5,238,652.

Alternative methods include the use of nucleic acid sequences such as oligonucleotides to detect the presence of a PCV nucleic acid in a biological sample.

For that purpose, one skilled in the art may use hybridization probes in solution hybridizations and in embodiments employing solid-phase procedures. In embodiments involving solid-phase procedures, the test is adsorbed or otherwise affixed to a selected matrix or surface. The fixed, single-stranded nucleic acid is then subjected to specific hybridization with selected probes.

In another embodiment, one skilled in the art may use oligonucleotide primers in an amplification technique, such as PCR (“polymerase chain reaction”), to specifically amplify the target PCV nucleic acid potentially present in the biological sample. Examples of such primers are given in Table 1 of Example 1.

### Prevention and Treatment of Congenital Tremors

The present invention contemplates vaccination or passive immunization to prevent or treat congenital tremors. The antigenic or immunogenic compositions of the invention are broadly applicable to protect a pig or its progeny from infection by porcine circovirus. The term “protect” is used herein to mean for the treatment or prevention of PCV infection, and congenital tremors. Thus, any animal susceptible to this type of infection can be vaccinated. Pigs may be treated at any age, and include new-born piglets. Treatment of sows is particularly useful to protect foetus.

The present invention more particularly relates to antigenic or immunogenic compositions that comprise a circovirus antigen, a veterinary acceptable vehicle or excipient and generally a veterinary acceptable adjuvant.

An immunogenic composition elicits an immunological response which can, but need not be, protective. A vaccine composition elicits a protective response. Accordingly, the term “immunogenic composition” includes a vaccine composition (as the former term can be protective composition).

The subject of the invention also is a method of immunization or of vaccination against congenital tremors, comprising the administration of an immunogenic composition or a vaccine against the porcine circovirus. This method of immunization or vaccination uses in particular the vaccines as defined below.

A subject of the present invention is thus an antigenic preparation directed against congenital tremors (CT), comprising at least one porcine circovirus (PCV) antigen. This antigen may consist of an attenuated live whole PCV, an inactivated whole PCV, a subunit antigen, a recombinant live vector, or a DNA vector.

### Whole PCV Vaccines

A subject of the invention is an isolated porcine circovirus strain, which has a genome comprising a nucleic acid sequence selected from the group consisting of any of ORF1 to ORF11 of any of the sequences of SEQ ID NO. 1 to SEQ ID NO. 7. Purified preparations of virions may be obtained by one skilled in the art knowing these sequences.

These virus may be used in an antigenic composition for vaccinating pigs against congenital tremors. For that purpose the virus particles may be attenuated, inactivated, or killed. according to standard techniques well known by one skilled in the art, as described below.

For the production of circovirus antigenic preparations, the circoviruses may be obtained after passage in cells, in particular cell lines, e.g. PK/15 cells. The culture supernatants or extracts, optionally purified by standard techniques, may be used as antigenic preparation.

In the context of attenuated antigenic preparations and attenuated immunogenic compositions or vaccines, the attenuation may be carried out according to the customary methods, e.g. by passage on cells, preferably by passage on pig cells, especially cell lines, such as PK/15 cells (for example from 50 to 150, especially of the order of 100, passages). These immunogenic compositions and vaccines comprise in general a veterinary acceptable vehicle or a diluent, with optionally in addition a veterinary acceptable adjuvant as well as optionally a freeze-drying stabilizer.

These antigenic preparations, immunogenic compositions and vaccines will preferably comprise from 10³ to 10⁷ TCID50 of the attenuated virus in question.

They may be antigenic preparations, immunogenic compositions and vaccines based on inactivated whole antigen. The inactivated immunogenic compositions and vaccines comprise, in addition, a veterinary acceptable vehicle or a diluent, with optionally in addition a veterinary acceptable adjuvant.

The circoviruses according to the invention, with the fractions which may be present, are inactivated according to techniques known to persons skilled in the art. The inactivation will be preferably carried out by the chemical route, e.g. by exposing the antigen to a chemical agent such as formaldehyde (formalin), paraformaldehyde, .beta.-propiolactone or ethyleneimine or its derivatives. The preferred method of inactivation will be herein the exposure to a chemical agent and in particular to ethyleneimine or to .beta.-propiolactone.

The antigenic preparations, immunogenic compositions and vaccines will preferably comprise from 10⁵ to 10⁸ TCID50 of the inactivated whole virus in question.

Preferably, the attenuated or inactivated antigenic preparations and the attenuated or inactivated immunogenic compositions and vaccines according to the invention will be supplemented with adjuvant, advantageously by being provided in the form of emulsions, for example water-in-oil or oil-in-water, according to techniques well known to persons skilled in the art. It will be possible for the adjuvant character to also come from the incorporation of a customary adjuvant compound into the active ingredient.

Among the adjuvants which may be used, there may be mentioned by way of example aluminium hydroxide, the saponines (e.g. Quillaja saponin or Quil A; see Vaccine Design, The Subunit and Adjuvant Approach, 1995, edited by Michael F. Powel and Mark J. Newman, Plennum Press, New-York and London, p.210), Avridine.RTM. (Vaccine Design p. 148), DDA (Dimethyldioctadecyl-ammonium bromide, Vaccine Design p. 157), Polyphosphazene (Vaccine Design p. 204), or alternatively oil-in-water emulsions based on mineral oil, squalene (e.g. SPT emulsion, Vaccine Design p. 147), squalene (e.g. MF59, Vaccine Design p. 183), or water-in-oil emulsions based on metabolizable oil (preferably according to WO 94/20071) as well as the emulsions described in U.S. Pat. No. 5,422,109. It is also possible to choose combinations of adjuvants, for example Avridine.RTM. or DDA combined with an emulsion.

As freeze-drying stabilizer, there may be mentioned by way of example SPGA (Bovarnik et al., J. Bacteriology 59, 509, 950), carbohydrates such as sorbitol, mannitol, starch, sucrose, dextran or glucose, proteins such as albumin or casein, derivatives of these compounds, or buffers such as alkali metal phosphates.

### Subunit and Vector Vaccines

As used herein, the term “subunit antigen” refers to an antigenic PCV polypeptide or an antigenic fragment thereof. The term “subunit or polypeptide vaccine” refers to a vaccine comprising an immunogenic polypeptide and, generally, an adjuvant.

A “vector vaccine” comprise “recombinant live vectors” or “DNA vectors”.

As used herein the term “recombinant live vector” refers to the vectors used to express an antigenic or immunogenic polypeptide for in vivo or ex vivo vaccination. Preferred vectors are viral vectors, such as DNA-based vectors and retroviral vectors. As appropriate live vectors, there may be used preferably live viruses, preferably capable of multiplying in pigs, nonpathogenic for pigs (naturally nonpathogenic or rendered as such), according to techniques well known to persons skilled in the art. There may be used in particular parvoviruses (U.S. Pat. No. 6,217,883), pig herpesviruses such as Aujeszky's disease virus, porcine adenovirus, poxviruses, especially vaccinia virus, avipox virus, canarypox virus, swinepox virus. DNA vectors can also be used as vectors (WO 90/11092,WO 93/19813,WO 94/21797, WO 95/20660). Generally, the vector is administered in vivo, but ex vivo transduction of appropriate antigen presenting cells, such as dendritic cells, with administration of the transduced cells in vivo, is also contemplated.

In another embodiment, the vector may be in the form of a DNA molecule that can be introduced in vivo by lipofection, as naked DNA, or with other transfection facilitating agents (peptides, polymers, etc.). This embodiment is herein referred to as the “DNA vector” technology. Synthetic cationic lipids can be used to prepare liposomes for in vivo transfection (Felgner, et. al., Proc. Natl. Acad. Sci. U.S.A. 84:7413-7417, 1987; Felgner and Ringold, Science 337:387-388, 1989; see Mackey, et al., Proc. Natl. Acad. Sci. U.S.A. 85:8027-8031, 1988; Ulmer et al., Science 259:1745-1748, 1993). Useful lipid compounds and compositions for transfer of nucleic acids are described in International Patent Publications WO95/18863 and WO96/17823, and in U.S. Pat. No. 5,459,127. Lipids may be chemically coupled to other molecules for the purpose of targeting (see Mackey, et al., supra). Targeted peptides, e.g., hormones or neurotransmitters, and proteins such as antibodies, or non-peptide molecules could be coupled to liposomes chemically. Other molecules are also useful for facilitating transfection of a nucleic acid in vivo, such as a cationic oligopeptide (e.g., International Patent Publication WO95/21931), peptides derived from DNA binding proteins (e.g., International Patent Publication WO96/25508), or a cationic polymer (e.g., International Patent Publication WO95/21931). It is also possible to introduce the vector in vivo as a naked DNA plasmid. Naked DNA vectors for gene therapy can be introduced into the desired host cells by methods known in the art, e.g., electroporation, microinjection, cell fusion, DEAE dextran, calcium phosphate precipitation, use of a gene gun (ballistic transfection), or use of a DNA vector transporter (see, e.g., Wu et al., J. Biol. Chem. 267:963-967, 1992; Wu and Wu, J. Biol. Chem. 263:14621-14624, 1988; Hartmut et al., Canadian Patent Application No. 2,012,311, filed Mar. 15, 1990; Williams et al., Proc. Natl. Acad. Sci. USA 88:2726-2730, 1991). Receptor-mediated DNA delivery approaches can also be used (Curiel et al., Hum. Gene Ther. 3:147-154, 1992; Wu and Wu, J. Biol. Chem. 262:4429-4432, 1987). U.S. Pat. Nos. 5,580,859 and 5,589,466 disclose delivery of exogenous DNA sequences, free of transfection facilitating agents, in a mammal. Recently, a relatively low voltage, high efficiency in vivo DNA transfer technique, termed electrotransfer, has been described (Mir et al., C.P. Acad. Sci., 321:893, 1998; WO 99/01157; WO 99/01158; WO 99/01175).

### Vaccination Strategies

Various strategies can be employed to vaccinate subjects against congenital tremors. The polypeptide vaccine formulations can be delivered by subcutaneous (s.c.), intraperitoneal (i.p.), intramuscular (i.m.), subdermal (s.d.), intradermal (i.d.), or by administration to antigen presenting cells ex vivo followed by administration of the cells to the subject.

Similarly, any of the gene delivery methods described above can be used to administer a vector vaccine to a subject, such as naked DNA and RNA delivery, e.g., by gene gun or direct injection.

Vaccination effectiveness may be enhanced by co-administration of an immunostimulatory molecule, such as an immunostimulatory or immunopotentiating, cytokine, lymphokine, or chemokine with the vaccine, particularly with a vector vaccine. For example, cytokines or cytokine genes such as interleukin (IL)-1, IL-2, IL-3, IL-4, IL-12, IL-13, granulocyte-macrophage (GM)-colony stimulating factor (CSF), macrophage inflammatory factor, as well as some key costimulatory molecules or their genes (e.g., B7.1, B7.2) can be used.

Mucosal Vaccination. Mucosal vaccine strategies are particularly effective for many pathogenic bacteria, since infection often occurs via the mucosa. Thus, mucosal vaccination strategies for both polypeptide and DNA vaccines are contemplated. While the mucosa can be targeted by local delivery of a vaccine, various strategies have been employed to deliver immunogenic proteins to the mucosa (these strategies include delivery of DNA vaccines as well, e.g., by using the specific mucosal targeting proteins as vector targeting proteins, or by delivering the vaccine vector in an admixture with the mucosal targeting protein).

For example, in a specific embodiment, the immunogenic polypeptide or vector vaccine can be administered in an admixture with, or as a conjugate or chimeric fustion protein with, cholera toxin, such as cholera toxin B or a cholera toxin A/B chimera (Hajishengallis et al, J Immunol.,154:4322-32, 1995; Jobling and Holmes, Infect Immun., 60:4915-24, 1992). Mucosal vaccines based on use of the cholera toxin B subunit have been described (Lebens and Holmgren, Dev Biol Stand 82:215-27, 1994). In another embodiment, an admixture with heat labile enterotoxin (LT) can be prepared for mucosal vaccination.

Other mucosal immunization strategies include encapsulating the immunogen in microcapsules (U.S. Pat. No. 5,075,109, No. 5,820,883, and No. 5,853,763) and using an immunopotentiating membranous carrier (WO 98/0558). Immunogenicity of orally administered immunogens can be enhanced by using red blood cells (rbc) or rbc ghosts (U.S. Pat. No. 5,643,577), or by using blue tongue antigen (U.S. Pat. No. 5,690,938). Systemic administration of a targeted immunogen can also produce mucosal immunization (see, U.S. Pat. No. 5,518,725).

Various strategies can be used to deliver genes for expression in mucosal tissues, such as using chimeric rhinoviruses (U.S. Pat. No. 5,714,374), adenoviruses, or specific targeting of a nucleic acid (WO 97/05267).

Passive Immunization

In addition to the active immunization vaccination strategies described above, the present invention further contemplates passive immunization with an antibody reactive with, and preferably generated against a PCV antigen. Passive immunization is particularly effective for an incipient or established infection, before the host's immune system can respond.

One source of antibodies for use in passive immunization is from convalescent serum of affected animals of the same species as the infected host. Thus, for example, antibodies from pig sera can be isolated, preferably by affinity purification against a PCV polypeptide and used to passively immunize newly infected pigs.

Alternatively, antibodies can be generated against the immunogenic polypeptide, i.e., the vaccine strategy can also be used to generate antibodies for passive immunization.

The anti-PCV antibodies of the invention may be cross reactive, e.g., they may recognize various PCV strains. Polyclonal antibodies have greater likelihood of cross reactivity.

### Immunoassay for Protective Immunity to Congenital Tremors

In another embodiment, an immunogenic polypeptide of the invention can be used in an immunoassay to detect protective antibodies against Congenital Tremors in a pig. Based on the discoveries of the present invention, a high titer of antibody reactive with (specific for) a PCV antigen indicates that the individual may be protected from an infection by PCV, and consequently from congenital tremors. Low or no detectable antibodies reactive with a PCV antigen indicates that the individual may not be protected from infection.

The immunoassay of the invention can be used to detect antibody levels in subjects who have been exposed to a PCV infection, e.g, in convalescent serum. It can also be used to detect antibodies in subjects of unknown status. High level antibody titers in such subjects would indicate prior exposure, and possibly protective immunity, to PCV. Finally, the immunoassay can be used to evaluate the effectiveness of a vaccine of the invention.

Any of the immunoassay formats described above can be used in an immunoassay of the invention. Preferably, an ELISA assay is used in which a PCV polypeptide is adsorbed to the solid phase, sera (preferably in serial dilution) is contacted with the solid phase, and antibody binding is detected, e.g., with a labeled antibody specific for antibodies in the serum. Alternatively, a competitive ELISA format could be used, in which anti-PCV antibodies in the serum sample compete for binding to the solid phase polypeptide against labeled antibodies specific for a PCV polypeptide, e.g., prepared as described above. In another alternative, the polypeptide is labeled, and antibody specific for the polypeptide adsorbed to the solid phase support. The presence of antibodies in the biological sample (e.g., serum) will result in competition for the polypeptide, preventing binding of the label to the adsorbed antibodies. In addition, convenient chromatographic immunoassay formats, as described below, can be used.

Although the immunoassays described here refer to testing for the presence of anti-PCV antibodies in serum, any biological sample that provides antibodies, can be tested, including without limitation, blood, serum, plasma, tissue samples, lymph, mucosal secretions, sputum, synovial fluid and other inflammatory fluids, and the like.

### Kits

The components for practicing the immunoassays can be conveniently provided in a kit form. In its simplest embodiment, a kit of the invention provides a PCV polypeptide and an antibody detector, such as a labeled antibody specific for antibodies from the subject to be tested. The amounts of each can be pre-measured to provide a specified number of assays.

In a further embodiment, the kit will include an assay container, such as a plate, preferably of plastic or a material treated to avoid non-specific binding of protein. As used herein, the term container has its broadest meaning, i.e., any receptacle for holding material or reagents. It can be fabricated from glass, plastic, ceramic, metal, etc.

In still a further embodiment, the kit includes an immunochromatographic membrane or support, to which one reagent, either a PCV polypeptide or an antibody specific for the PCV polypeptide has been irreversibly coupled. Numerous methods and devices known in the art for immunochromatographic assays can be employed in the invention. As noted above, immunochromatographic assays are particularly useful under field conditions, where laboratory equipment is not available. Examples of such assays are provided in U.S. Pat. No. 5,248,619, No. 5,451,504, No. 5,500,375, No. 5,624,809, and No. 5,658,801.

A kit of the invention preferably includes packaging and instructions for its use, e.g., on the packaging or package insert.

### Combined Strategies

In the context of combined immunization or vaccination programs, it is also possible to combine the immunization or vaccination against the porcine circovirus with an immunization or vaccination against other pig pathogens, in particular those which could be associated with the PMWS syndrome. The immunogenic composition or vaccine according to the invention may therefore comprise another valency corresponding to another pig pathogen chosen from parvovirus (U.S. Pat. No. 6,217,883), from PRRS (Porcine Reproductory and Respiratory Syndrome) and/or Mycoplasma hyopneumoniae, and/or E. coli, and/or Atrophic Rhinitis, and/or Pseudorabies (Aujeszky's disease) virus and/or porcine influenza and/or Actinobacillus pleuropneumoniae and/or Hog cholera, and combinations thereof. Preferably, the programme of immunization or vaccination and the vaccines according to the invention will combine immunizations or vaccinations against the circovirus, and the PRRS (WO 93/07898, WO 94/18311, FR-A-2 709 966; C. Charreyre et al., Proceedings of the 15.sup.th IPVS Congress, Birmingham, England, Jul. 5-9, 1998, p 139; and/or Mycoplasma hyopneumoniae (EP-A-597 852, EP-A-550 477, EP-A571 648; 0. Martinon et al. p 157, 284, 285 and G. Reynaud et al., p 150, all in the above-referenced Proceedings of the 15.sup.th IPVS Congress) and/or porcine influenza. It is thus possible to use any appropriate form of immunogenic composition or vaccine, in particular any available commercial vaccine, so as to combine it with the immunogenic composition or vaccine against the porcine circovirus as described here.

The subject of the present invention is therefore also multivalent immunogenic compositions and vaccines, multivaccine kits, and combined immunization or vaccination methods which makes it possible to use such combined immunization or vaccination programs.

The present invention will be better understood by reference to the following examples, which are provided by way of exemplification and are not intended to limit the invention.

### EXAMPLES

### Example I

### Sequence Analysis of Porcine Circovirus Associated with Congenital Tremors in Pigs.

### Methods

Tissue samples and a cell line infected with PCV Pigs infected with PCV were obtained from farms in Indiana, USA. Pigs were initially identified by signs of either PMWS or CT, followed by microscopic examination of tissue sections. Presence of PCV in tissue was further confirmed by in situ hybridization using PCV-specific oligonucleotide probe, indirect fluorescent assay (IFA) using antiserum against PCV and PCR using primers specific to PCV. Four PCV isolates collected from pigs showing signs of PMWS were named as PMWS-PCV-P 1, PMWS-PCV-P2, PMWS-PCV-P3, and PMWS-PCV-P4. Two PCV isolates collected from pigs showing signs of CT were named as CT-PCV-P5, and CT-PCV P6.

For the isolation of a historic PCV isolate, a PCV contaminated cell line (PCNS) was derived from the brain of a pig showing signs of CT. A pregnant sow was experimentally inoculated with the cell culture supernatant from CT pig kidney cells (Gustafson & Kanitz, 1974). PCNS cells were grown in Eagle's minimum essential medium (EMEM) [Life Technologies, Inc.] containing 10% FetalClone III (HyClone, Inc.). Cells were harvested and tested for PCV by in situ hybridization using a PCV-specific oligonucleotide probe, electronmicroscopy (EM), and by PCR using PCV-specific primers. This PCV isolate was named as CT-PCV-P7.

DNA isolation and PCR. PCNS cells grown in EMEM were harvested when cells started floating in the medium. The cell pellet was lysed by SDS-pronase (500 μg/ml pronase in 10 mM Tris, pH 7.4, 10 mM EDTA, and 0.5% SDS) and incubated at 37° C. overnight. The total cellular DNA was isolated by phenol extraction followed by ethanol precipitation.

Lymph nodes for PMWS-PCV-P I, -P2, -P3, -P4, and CT-PCV-P6 and liver for CT-PCV-P5 were homogenized in EMEM using a tissumizer followed by sonication using a sonicator. Tissue homogenates were incubated with the equal volume of SDS-pronase (1 mg/ml pronase in 20 mM Tris, pH 7.4, 20 mM EDTA, and 1% SDS) at 37° C. overnight. Total cellular DNA was obtained by phenol extraction and ethanol precipitation. The DNA was used as a template for PCR using Vent DNA polymerase (New England BioLab) with two pairs of primers to amplify the entire PCV genome. For PMWS-PCV-P I, -P2, and -P3, CT-PCV-P5, and -P6; PCV2-1 & PCV2-2 and PCV23 & PCV2-4 sets of primers were used (Table 1). For PMWS-PCV-P4, PCV2-1 & PCV2-2 and PCV4-1 & PCV4-2 sets of primers were used (Table 1). For CT-PCV-P7, PCV1-3 & PCV1-4 and PCV7-1 & PCV7-2 sets of primers were used (Table 1). PCR products were analyzed on 1% agarose gel and visualized with an UV transilluminator.

TABLE 1
Nucleotide sequence and the location of primers
used in this study for the cloning of PCV genome

| | | Location of primer | |
|---|---|---|---|
| Name of primer | SEQ ID NO | (nucleotide)^{a} | Nucleotide sequence of primer |
| PCV1-3 | 8 | 37-56 | 5′TACTCCTCAACTGCTGTCCC3′ |
| PCV1-4 | 9 | 1605-1624 | 5′TCCATCCCACCACTTATTTC3′ |
| PCV2-1 | 10 | 1076-1093 | 5′ACGCTGAATAATCCTTCC3′ |
| PCV2-2 | 11 | 679-660 | 5′CCAACAAAATCTCTATACCC3′ |
| PCV2-3 | 12 | 7-24 | 5′ATTACCAGCAATCAGACC3′ |
| PCV2-4 | 13 | 1657-1640 | 5′AACAACCACTTCCTTCACC3′ |
| PCV4-1 | 14 | 611-629 | 5′AGCAGGGCCAGAATTCAAC3′ |
| PCV4-2 | 15 | 1100-1079 | 5′CGTCTTCGGAAGGATTATTCAG3′ |
| PCV7-1 | 16 | 1597-1617 | 5′GCCTAGTAGAAATAAGTGGTG3′ |
| PCV7-2 | 17 | 87-68 | 5′AGTAATCCTCCGATAGAGAG3′ |

^{a}The location of primers PCV2-1. 2-2, 2-3, 2-4, 4-1, 4-2 and primers PCV I-1, 1-2, 7-1, 7-2 is based upon the sequence of PMWS-PCV (Hamel et al.; 1999) and PK-1 5-PCV (Meehan et al. 1997). respectively.

Cloning of PCR products. The PCR products were cloned into the SmaI site of pUC 18 by blunt-end ligation using T4 DNA ligase (New England Bio Lab). To construct the entire genome of PMWS-PCV-P I, -P2, and-P3, pUC 18 containing PCR products from nt 1076-679 amplified with PCV2-1 & 2 primers and PCR products from nt 7-1657 amplified with PCV2-3 & 4 primers were digested with StuI and KpnI. The 4 kb StuI-KpnI fragment from pUC 18 containing PCV2-1 & 2 amplified PCR product was used to insert a 1.3 kb StuI-KpnI fragment from pUC18 containing PCV2-3 & 4 amplified PCR product to result in pUC 18 containing PCV genome from nucleotide 1076-1768 and 1-1657. The resultant plasmids containing the genome of either PMWS-PCV-P I, -P2 or -P3 were named pPCV-PI, pPCV-P2 and pPCV-P3, respectively. These plasmids when digested with SacII 135 produced the linearized-form of complete PCV genomes. Similarly PCR products obtained from other PCV strains were also cloned at the SmaI site of pUC18 by blunt-end ligation. Plasmid DNA was purified by isopycnic centrifugation in cesium chloride-ethidium bromide gradients (Sambrook et al., Molecular cloning: A laboratory manual. Cold Spring Harbor Laboratory Press, 1989).

Transfection of cloned PCVDNAs and detection ofPCV Plasmids containing entire genome of PCV DNA (PPCV-P 1,-P2, and -P3) were digested with SacII to result in two fragments, the full-PCV genomic DNA and pUC 18 plus a part of PCV DNA. Semi-confluent monolayers of PCV-free PK-15 cells in 6-well plates were transfected with 1 μg of the ligated PCV genome using Lipofectin-mediated transfection protocol (Life Technologies, Inc.). Cells were passaged three times. After the third passage, cells were harvested, cytospined and fixed with acetone. Polyclonal antibody against PMWS-PCV raised in a rabbit (Morozov and Paul, Iowa State University, Ames, Iowa) was used for IFA. For EM, water was added to cell pellets and the cell contents were centrifuged at 10,000 RPM for 5 min. Supernatants were collected and centrifuged at 20,000 RPM for 40 min. The cell pellets were resuspended in water containing 3% phosphotungstic acid and 1% bovine serum albumin. Samples were nebulized onto the carbon-coated grids and examined with a Philips 201 electronmicroscope.

DNA sequencing and sequence analysis. Plasmids containing PCV DNA were sequenced using universal and reverse primers. Subsequently, both strands of DNA were sequenced by primer walking using an applied Biosystems 373A automated sequencer. The entire genomes of 7 PCV isolates (PMW-PCV-P1, -P2, -P3, -P4, and CT-PCV-P5, -P6, and -P7) were analyzed using the GCG sequence analysis software (Wisconsin package).

Phylogenetic calculations. The sequence alignments were gained by the ClustalW program. The phylogenetic calculations were performed by the PHYLIP program package version 3.572c (Felsenstein, Cladistics 5:164-166, 1989). For the parsimony analysis, the programs Protpars or DNApars were used. For the distance analysis, the Protdist (Dayhoff's PAM 001 matrix) or DNAdist (Kimuia 2-parameter) followed by Fitch (with global rearrangements) were applied. During the bootstrap analysis, the above calculations were preceded by the Seqboot (100 datasets) and followed by the Consensus program to get the consensus tree. Finally, the results were visualized by the TreeView program (Page, Computer Applications in the Biosciences 12:357-358, 1996). A more detailed description of the applied methods has been published elsewhere (Harrach & Benkö, Adenovirus Methods and Protocols Methods in Molecular Medicine, 21:309-339, 1998).

### Results

Transfection of PCV-free PK-15 cells with PCV DNAs. Initially, the entire genomes of three PCV isolates associated with PMWS (PMWS-PCV-P1,-P2, and -P3) were amplified by PCR using two sets of primers. PCR generated fragments were used to construct plasmids (pPCV-P 1, -P2 and -P3) containing PCV genomes. These plasmids, on digestion with SacII, resulted in a linear-form of complete PCV genome. To test whether the cloned PCV genomes were infectious, the SacII-digested-religated or unligated pPCV-P 1, -P2 and -P3 were used to transfect PCV-free PK-1 5 cells. Cells were harvested after third passage and analyzed for the presence of PCV antigen by IFA. A number of cells transfected with SacII-digested religated PCV DNA were positive for PCV antigen, whereas cells transfected with SacII-digested unligated PCV DNA were negative for PCV antigen by IFA. To further determine whether transfection with PCV DNA resulted in the production of PCV virion, PK-15 cells transfected with PCV DNA were analyzed by EM. Small spherical viruses, approximately 17 nm in diameter, were observed. The detection of PCV antigen and the observation of virus particles in cells transfected with PCV DNA indicated that the cloned full-length circular PCV DNA results in viral replication and production of virus particles. Since the full-length PCV genomes amplified by PCR were infectious, the inventors used PCR technique to amplify genomes of other PCV field strains.

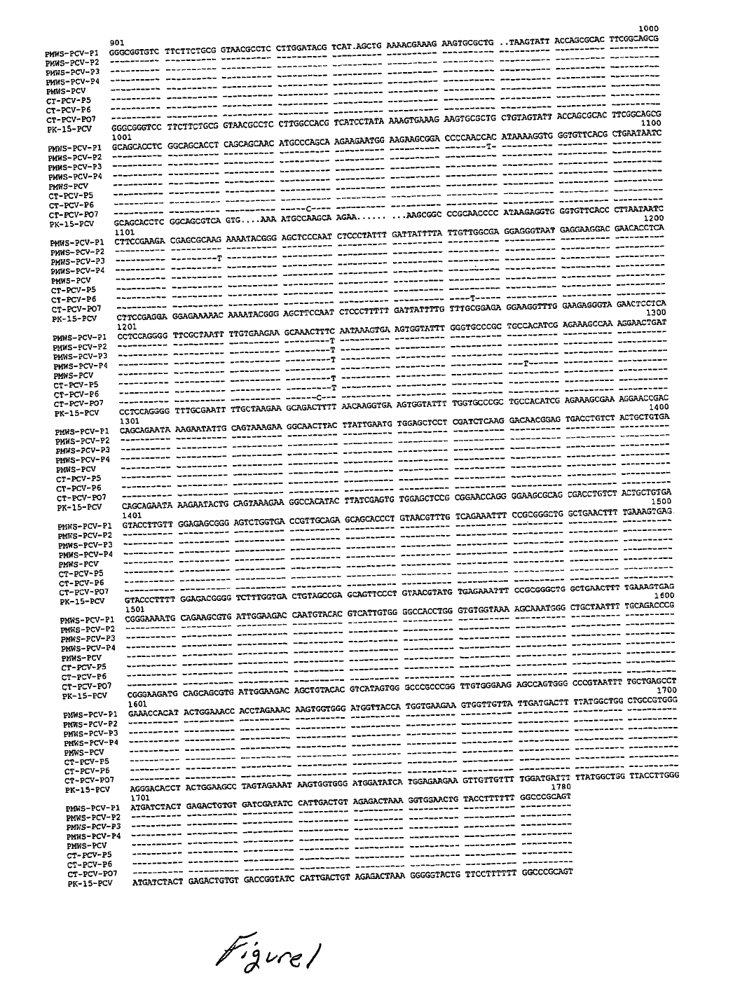

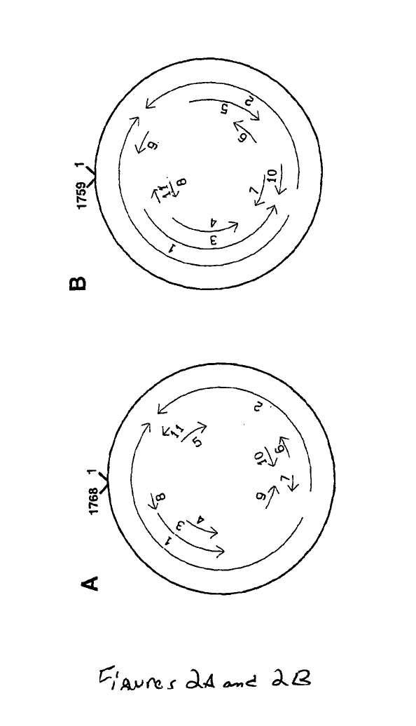

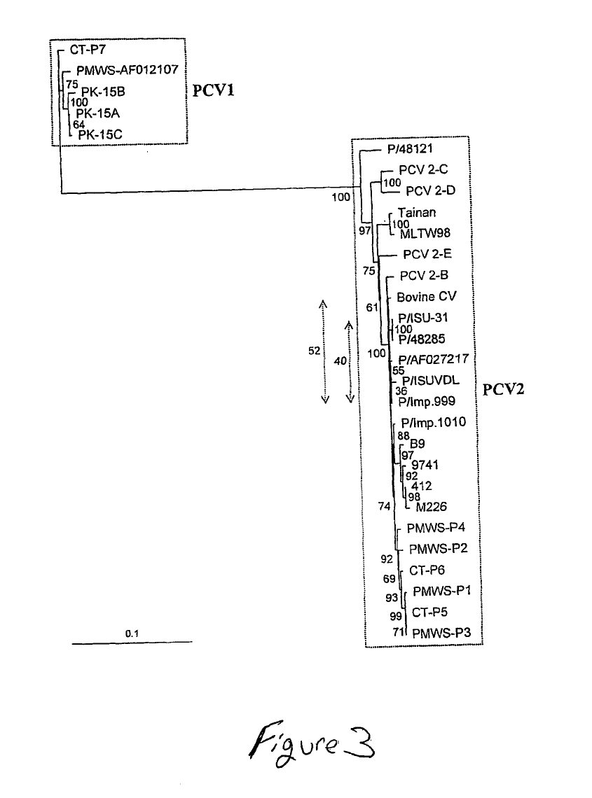

Sequence comparison of PMWS-PCV, and old and new CT-PCV isolates. The inventors sequenced the entire genomes of 4 PCV isolates associated with PMWS (PMWS-PCV P 1, -P2, -P3 and -P4), 2 PCV isolates associated with CT in the late 1990s (CT-PCV-P5 and -P6)), and one PCV isolate associated with CT in the late 1960s (CT-PCV-P7). Sequences of these isolates were compared with that of the previously described PMWS-PCV isolate (Hamel et al., 1998) and PK-1 5-PCV isolate (Meehan et al., Journal of General Virology 78:221-227, 1997). Genomes of PMWS-PCV-PI, -P2, and -P4 were 1768 nucleotides (nt) long, whereas PMWS-PCV-P3 was 6 nucleotides shorter than the rest of PMWS PCV isolates due to a 6-nt deletion between 820 to 825 nt (FIG. 1). All the PMWS-PCV isolates of the invention had an overall 99% nt sequence identity with each other. The orientation and the relative length of each ORF of PMWS-PCV-PI are shown (FIG. 2A). The coding strand, number of amino acids, and the location of each ORF in the genomes of PMWS-PCVs are listed (Table 2).

TABLE 2
Comparison of ORFs of PMWS-PCVs and new CT-PCVS*

| | PMWS-PCV-P1 | | PMWS-PCV-P2 | | PMW-PCV-P4 | | |
|---|---|---|---|---|---|---|---|
| | Coding | Number of | Location | Number of | Location | Number of | Location |
| ORF | strand | amino acids | (nucleotide) | amino acids | (nucleotide) | amino acids | (Nucleotide) |
| ORF1 | V | 314 | 1019-195 | — | 1013-195 | — | — |
| ORF2 | C | 233 | 935-234 | 231 | 926-234 | — | — |
| ORF3 | C | 104 | 1639-1325 | — | 1633-1319 | — | — |
| ORF4 | C | 59 | 1533-1354 | — | 1527-1348 | — | — |
| ORF5 | V | 53 | 216-377 | — | — | 104 | 216-530 |
| ORF6 | C | 29 | 811-724 | — | — | — | — |
| ORF7 | V | 19 | 882-941 | — | 876-935 | — | — |
| ORF8 | C | 21 | 1721-1656 | — | 1715-1650 | — | — |
| ORF9 | C | 42 | 1061-932 | — | 1055-926 | — | — |
| ORF10 | V | 35 | 724-931 | 61 | 724-909 | — | — |
| ORD11I | C | 14 | 233-189 | — | — | — | — |

*Number of amino acids and the location of each ORF of PMWS-PCV-P3, CT-PCV-P5, and CT-PCV-P6 are identical to those of PMWS-PCV-P1.
^{a}V indicate viral strand that is encapsidated into virus particles and C indicates the complementary strand to viral strand.
— Indicates same as the corresponding of PMWS-PCV-P1I

The amino acid sequence of ORF1 was highly homologous (approximately 99% homology at amino acid level) among all these PMWS-PCV isolates. Observed changes in amino acid residues in various ORFs of these PMWS-PCV isolates are listed (Table 3). The ORF2 had more amino acid changes than ORF1 among PMWS-PCVS, but still had an approximately 97% homology. Open reading frames 3, 4, 7, and 8 were identical among PMWS-PCV isolates and there were only few changes in the rest of ORFs (Table 3).

TABLE 3
Amino acid sequence comparison of ORFs of various PCVs with those of
PMWS-PCV-P1 or PK-15-PCV*

| | PMWS-PCV- | PMWS-PCV- | PMWS-PCV- | | | |
|---|---|---|---|---|---|---|
| | P2 | P3 | P4 | CT-PCV-P6 | PMWS-PCV^{b} | CT-PCV-P7 |
| ORF1 | | K30N, T292M | | | H82Y | T283N |
| ORF2 | P, 59^{a}, K75T. | R35dY36d | R59A, K75N. | N134T. N2-32K | R59A, T63F, | A30V, T44K, |
| | L761, V130F, | | L76L P13IT, | | K75N, L761, | T53R, Y63H |
| | A133S, | | N134S. | | P13IT, N134T, | Y72H, K74R, |
| | N134T, | | L185K L1871, | | N181T, K206L | H176Q, |
| | N232K | | N2S2K | | N232K | Y201F, |
| | | | | | | A207D, K233E |
| ORF3 | — | — | — | | — | S139G, |
| | | | | | | A163T, |
| | | | | | | W203G |
| ORF4 | — | — | — | | — | — |
| ORF5 | F51, V9F | — | F5I, V9F, | F51, V9F | V9F, F35Y | V12L, N69Y |
| | | | H48Y | | | |
| ORF6 | S17P, V18L | P25R | S17RV18L | A7G, S8F | S17R, VI8L, | N6D, Q16R, |
| | | | | | Q22E | L51stop |
| ORF7 | — | — | — | | | K48Q |
| ORF8 | — | — | — | — | — | — |
| ORF9 | W2R | — | — | — | — | Q25H |
| ORF10 | R13A | Q5H | R13A | R23S | V9L, R13A | — |
| ORF11 | K2N | — | K2N | K2N | — | — |

*ORFs of PMWS-PCV-P2, -P3, -P4, PMWS-PCV, and CT-PCV-P6 were compared with those of PMWS-PCV-1, whereas ORFs of CT-PCV-P7 were compared with those of PK-15-PCV.
^{a}Amino acid change is depicted as amino acid present in the PMWS-PCV-P1, the location and the changed amino acid.
^{b}The sequence of PMWS-PCV has been published (Hamel et al., Journal of Virology 72:5262-5267 1998).
^{d}Indicates the deleted amino acid.
Stop: Indicates the stop codon resulting in the end of ORF.

The ORFs of PMWS-PCV-P4 and ORF10 of PMWS-PCV-P3 were 54 and 26 amino acids longer, respectively than their counterpart in the rest of our PMWS-PCV isolates.

Two new CT-PCV isolates (CT-PCV-P5 and -P6), which were isolated in the late 1990s, were 1768 nt long (FIG. 2). These CT-PCVs had approximately 99% nt sequence identity. Interestingly, new CT-PCV isolates also demonstrated an approximately 99% nt sequence identity with the new PMWS-PCV isolates. The genomes of PMWS-PCV-P1 and CT-PCV-P5 were identical. Both PMWS-PCV and new CT-PCV genomes encode 11 potential ORFs. The amino acid changes in the various ORFs of new CT-PCV compared to those of PMWS-PCV-P1 are listed (Table 3).

The genome of the old CT-PCV (CT-PCV-P7) was 1759 nucleotides long (FIG. 1). The CT PCV-P7 genome also encoded 11 potential ORFs. The orientation and relative length of each ORF are shown in FIG. 2B.

The numbers of amino acids and the location of each ORF of CT-PCV-P7 are shown (Table 4).

TABLE 4
Comparison of ORF of PK-15-PCV and old CT-PCV

| | | PK-15-PCV | | CT-PCV-P7 | |
|---|---|---|---|---|---|
| | | Number of | | Number of | |
| | Coding | amino | Location | amino | Location |
| ORF | Strand^{a} | acids | (nucleotide) | acids | (nucleotide) |
| ORF1 | V | 312 | 1019-198 | — | — |
| ORF2 | C | 233 | 936-235 | — | — |
| ORF3 | C | 206 | 1630-1010 | — | — |
| ORF4 | C | 115 | 1524-1177 | — | — |
| ORF5 | V | 95 | 376-663 | — | — |
| ORF6 | C | 62 | 731-543 | 50 | 731-579 |
| ORF7 | V | 56 | 883-1053 | — | — |
| ORF8 | C | 37 | 1712-1599 | — | — |
| ORF9 | C | 31 | 181-86 | — | — |
| ORF10 | V | 37 | 855-968 | — | — |
| ORF11 | V | 23 | 1620-1691 | — | — |

^{a}V indicates viral strand that is encapsidated into virus particles and C indicates the complementary strand to viral strand.
— Indicates same as the corresponding of PK-15-PCV.

The genome of CT-PCV-P7 had only approximately 72% nt sequence identity with PMWS-PCVs and both new CT-PCVS, but shared a surprising approximately 98% nt sequence identity with PK-15-PCV. Amino acid sequences of all ORFs of CT-PCV-P7 were also highly homologous to those of PK-15-PCV. Amino -acid changes in various ORFs of CT-PCV-P7 compared to their counterpart in PK-15-PCV are listed (Table 3).

Meehan et al., J Gen Virol 78:221-227, 1997 observed the presence of a nonanucleotide sequence at the apex of the stem loop structure of PK-15-PCV which was similar to that described in nanoviruses and geminiviruses of plants. All our PCV isolates had the conserved stem-loop structure and nonanucleotide (A/TAGTATTAC), representing the origin of rolling-circle DNA replication (Mankertz, et al., Journal of Virology 71:2562-2566, 1997; Journal of General Virology 79:381-384, 1998). The potential glycosylation sites (N-X-T or N-X-S, where ‘X’ is any amino acid), which were previously reported by Hamel et al., (1998), were conserved in all our PCV isolates except ORF6 of CT-PCV-P7 where the amino acid residue ‘N’ at number 6 was replaced with ‘D’.

Phylogenetic analysis. When either the deduced amino acid sequences of individual proteins, such as the replication associated protein (ORF1/replicase/rep/coat/P35.8 protein) and the protein P27.9 (ORF2), or the nucleotide sequence of the full genome were used for phylogenetic analysis, both the distance matrix and the parsimony analyses yielded two distinct clusters of fairly similar topology. Since the differences between the strains were moderate, and the distance matrix analysis seemed to yield the more consistent data (Harrach & Benkö, Adenovirus Methods and Protocols Methods in Molecular Medicine, 21:309-339, 1998), the inventors chose to present their findings by the distance matrix analysis of the full genomes (FIG. 3).

The most evident result of the distance matrix analysis of various porcine and bovine circovirus genomes was the clear separation of the isolates into two clusters (FIG. 3). No intermediate genotypes were found though the number of the examined genomes (including the 7 new sequences) included 29, and the origin of the isolates covered geographically distant regions.

The smaller cluster (type 1) contained the isolates from the different lineages of the PK-15 cell line, and 2 circovirus strains (PMWS accession number AF012107 and CT-PCV-P7) isolated from different pathological entities (PMWS and CT). The other fairly large cluster (type 2). contained the remaining 24 isolates, including 21 from pigs with PMWS, the 2 recent CT isolates (CT-PCV-P5 & -P6) and a bovine isolate (AF109397). Based on this phylogenetic tree, the genetic relatedness of the circovirus strains seems not to be directly connected to the pathogenic ability.

### Discussion

The goal of this study was to determine genetic variability in PCV associated with CT. The PMWS-PCV isolates yielded an approximately 99% nt sequence identity with each other and also 96% nt identity to PMWS-associated PCVs isolated in the U.K., Canada, France, and U.S. (Meehan et al, Journal of General Virology 79:2171-2179, 1998; Morozov et al., Journal of Clinical Microbiology 9:2535-2541, 1998; Hamel et al, Journal of Virology 72:5262-5267, 1998; Mankertz et al. Virus Research, 6665-77, 2000) indicating that various PMWS-PCV isolates are highly homologous regardless of their place of origin.

Although the new CT-PCV isolates from the late 1990s and the old CT-PCV isolate from the late 1960s originated from neonatal pigs with CT type A2, they shared only 72% nt sequence identity. The genomes of the 2 new CT-PCVs were rather similar to the recent isolates of PMWS PCV, whereas the old CT-PCV isolate was very close to PK-15-PCV variants. Based on extensive phylogenctic calculations, different PCV isolates can be divided into 2 groups. PK-15-PCV variants, our old CT-PCV-P7 and a single uncharacterized PMWS isolate (AF012107) comprise PCV type I (PCV1) and the remaining 20 recent PMWS-PCVs and the 2 new CT-PCV field isolates (CT-PCV-P5 & -P6) comprise PCV type 2 (PCV2). On the basis of sequence analysis of a PK-15-PCV and 4 PMWS associated PCV isolates, a preliminary proposal to classify PK-15-PCV as PCV 1 and PMWS-PCV isolates as PCV2 was suggested (Meehan et al., 1998).

PK-15-PCV (a PCV 1 isolate) was clinically nonpathogenic in inoculation studies in weaned pigs (Tischer et al., Archives of Virology 91:271-276, 1986; Allan et al, Veterinary Microbiology 44:49-64, 1995), whereas the CT-PCV-P7 isolate (also a PCV1) was derived from a neonatal pig with CT in the late 1960s and seemingly caused congenital tremors in progeny when inoculated into a pregnant sow at 70 days-of-gestation (Kanitz, Ph.D. Dissertaion, Purdue University, 1972). It is unclear whether PK-15-PCV could also cause CT or whether CT-PCV-P7 is pathogenic in weaned pigs. The age, route of infection and/or some other factors may determine the pathogenicity and clinical manifestations of PCV 1 and PCV2. The presence of an approximate 99% nt sequence identity among the new CT-PCV and PMWS-PCV strains indicates that recent outbreaks of PMWS and CT are associated with the same type of PCV (i.e., PCV2). The reported age of pigs with PMWS is 6-12 weeks (Ellis et al., Canadian Veterinary Journal 39:44-51, 1998; Kiupel et al., Indiana.Veterinary Pathology 35:303-307, 1998; Rosell et al., Journal of Comparative Pathology 120:59-78, 1999), whereas CT is a disease of newborn pigs (Stevenson et al., Journal of Veterinary Diagnostic Investigations, in press). It appears that age plays a critical role in determining the type of syndrome caused by PCV. The presence of PCV2 DNA has recently been demonstrated in large numbers of neurons in brain and spinal cord of neonatal pigs with naturally occurring CT. Neural cell division occurs exclusively during fetal development. Thus, during fetal development may be the only period when PCV could replicate in nervous tissues leading to signs of CT, because PCV requires cell division for its replication (Tischer, et al., Archives of Virology 96:39-57, 1987). When PCV2 is inoculated into germ-free pigs, lesions but not clinical disease typical of PMWS develop by 35 days postinoculation. However, when PCV2 is inoculated with porcine parvovirus or porcine reproductive and respiratory syndrome virus, replication of PCV is enhanced and PMWS is reproduced (Allan et al., Journal of Comparative Pathology 121, 1-11, 1999). Other viruses might enhance PCV replication by directly or indirectly causing division of PCV-target cells.

PCV1 was first identified during the 1960s and 70s (Tischer et al., Zentralblatt fur Bakteriologie, Parasitenkunde, Infektionskrankheiten, und Hygiene—Erste Abteilung Originale—Reihe A: Medizinische Mikrobiologie und Parasitologie 226:153-167, 1974; Meehan et al, Journal of General Virology 78:221-227, 1997; this study), whereas PCV2 was identified in the late 1990s (Hamel et al., Journal of Virology 72:5262-5267, 1998; Meehan et al., Journal of General Virology 79:2171-2179, 1998; Mankertz et al., Virus Research, 6665-77, 2000). On the basis of sequence analysis, it appears that PCV2 may have derived from PCV 1. However, the large phylogenetic distance between the two types and the seeming total lack of intermediates contradicts a direct and recent connection between the two types. These findings do not support a role of the PK-15 cell line as origin of a wide spread PCV infection (e.g., vaccine borne disease).

The cluster of type 2 PCVs includes a single bovine-origin circovirus isolate. It is unknown how widespread circoviral infection is in bovids. Based on the high similarity between this single bovine isolate and PCV2's, the tempting speculation that the two different PCV lineages previously and simultaneously evolved in porcine and bovine hosts as seen in the case of adenoviruses (Russell & Benko, Encyclopedia of Virology, pp. 14-21, 1999) is contradicted. However, such PCV1 and PCV2 strain evolution may have occurred in 2 or more yet unidentified host species.

### Example II

### Tissue Distribution and Genetic Typing of Porcine Circoviruses in Pigs with Naturally Occurring Congenital Tremors

### Materials and Methods

Study design . Pigs less than 2 days-of-age were selected from 4 farms in the Midwestern United States that were experiencing outbreaks of disease consistent with CT type A2. From each farm, 2-4 pigs with CT (n=13) and 1-2 clinically normal pigs (n=6) were transported to Purdue Animal Disease Diagnostic Laboratory, Lafayette, Ind. where they were euthanized with pentobarbitol. Necropsy examinations were performed and tissues were collected for testing. Samples of cerebrum, cerebellum, pons, spinal cord segments C1, C4, C7, T3, T6, T9, T12, L2, L5 and S2, lung, liver, kidney, spleen, tonsil, mesenteric and inguinal lymph nodes were collected in neutral buffered formalin or frozen at −20° C. for testing.

Histopatliology and in-situ hybridization. Tissues were fixed at room temperature for 24 hours then were embedded in paraffin, sectioned and stained with hematoxylin and eosin by routine methods. All tissues were evaluated for microscopic lesions. In situ hybridization was accomplished using a PCV oligonucleotide probe known to hybridize with both PCV1 and PCV2 as previously described (Kiupel et al., Eur J Vet Pathol., 1999; Rossell et al., Encyclopedia of Virology, pp. 14-21, 1999). The PCV-specific oligonucleotide was 3′-end labeled with digoxigenin (Boehringer Mannheim Biochemica, Indianapolis, Ind.). After deparaffinization, proteolytic digestion with 0.25% pepsin for 8 min at 105° C. followed by 10 min at 37° C., washes in automation buffer and prehybridization with 100% formamide for 5 min at 105° C., hybridization was performed for 5 minutes at 105° C. and 60 minutes at 37° C. with a probe concentration of 5 μl/ml using a commercial workstation (Fisher Scientific, Pittsburgh, Pa.). High stringency washes were made with saline sodium citrate buffer to ensure binding of probe and target. The detection system consisted of the antidigoxigenin antibody conjugated with alkaline phosphatase (dilution 1:500) (Boehringer Mannheim Biochemica, Indianapolis, Ind.) applied at 37° C. for 45 min and the substrates “NBT/X-Phos” (Nitro-blue tetrazoliun 5-Bromo-4-chloro-3-indolylphosphate) (Boehringer Mannheim Biochemica, Indianapolis, Ind.). Dye reduction to insoluble blue formazan was allowed for 45, 90 and 180 min on serial sections. Controls included dot-blot slides of PCV 1-infected PK-15 Cells (Stevenson et al., Veterinary Pathology 36:368-378, 1999) brain, spinal cord, and lymphoid tissue from PCV2-infected Pig (Kiupel et al., Indiana.Veterinary Pathology 35:303-307, 1998) and from PCV-free gnotobiotic pigs. Slides incubated with hybridization solution without probe were used as negative reagent controls.

Polymerase chain reaction (PCR) testing. PCR testing of cerebellum and liver samples from all pigs was accomplished to determine the genotype of PCV as type 1 or type 2. Controls were the same as used for in situ hybridization testing. Tissues were homogenized in equal volume of TE (10 mM Tris-Hcl, 1 mM EDTA, pH 7.5) using a tissumizer. Total cellular DNA was extracted using a standard protocol (Sambrook et al., Molecular Cloning: A Laboratory Manual. Cold Spring Harbor, 1989). Primer sets were designed to be specific for PCV (Meehan et al., Journal of General Virology 78:221-227, 1997) or PCV2 (Hamel et al., Journal of Virology 72:5262-5267, 1998) and were used to amplify PCV sequences by PCR using Vent DNA polymerase (New England Biolab, Inc., Beverly, Mass.). PCR amplified DNA samples were analyzed on 1% agarose gel by electrophoresis and the bands of the expected size were visualized with an UV transilluminator. Specificity of PCR results was confirmed by sequence analysis.

Frozen-section indirect immunofluorescent antibody testing. Samples of liver and cerebellum that had previously tested positive for PCV by in-situ hybridization were selected from one pig from each herd. Indirect fluorescent antibody testing was completed to confirm the presence of PCV-specific antigen. Frozen tissue sections were prepared and indirect fluorescent antibody tests were performed by routine methods using a commercially available polyclonal antibody (Morozov and Paul, Iowa State University, Ames, Iowa) produced against purified PCV2 raised in a rabbit at a 1:500 dilution and fluorescein-conjugated murine anti-rabbit IgG at 1:250 dilution.

Testing for other agents. Routine testing using indirect immunofluorescence for other swine viral agents, including pseudorabies virus NYSL, Ames, Iowa), swine influenza viruse (NYSL, Ames, Iowa), porcine rotavirus (NYSL, Ames, Iowa), porcine hemagglutinating encephalomyelitis virus (NYSL, Ames, Iowa), porcine parvovirus (American Bioresearch, Sevierville, Tenn.) and transmissible, gastroenteritis virus (American Bioresearch, Sevierville, Tenn.) was completed at Purdue Animal Disease Diagnostic Laboratory, Lafayette, Ind. using commercially available diagnostic tests.

Samples of serum, spleen and lung were tested by virus isolation in swine primary alveolar macrophage cell cultures for porcine reproductive and respiratory syndrome (PRRS) virus.

Samples of brain, spleen and tonsil were tested by direct fluorescent antibody tests and virus isolation in swine turbinate cells for pseudorabies virus. Samples of brain, spleen and tonsil were tested in swine turbinate and swine testicular cells for cytopathic viruses.

### Results

All 4 farms experiencing outbreaks of CT purchased all replacement breeding stock from outside sources. Sources of breeding stock were different for each farm and no farms shared common genetics. On the three farms, where pigs were retained to slaughter age, there was no recent history of PMWS. All pigs with CT that were selected for study were ≦48 hours old and had moderate to severe tremors that were most severe when pigs attempted voluntary movements. Tremors partially abated when pigs rested. All pigs selected as age-matched, clinically normal, control pigs originated from lifters with no CT pigs. All pigs selected for testing were alert, active and otherwise clinically normal.

There were no gross or microscopic lesions in any CT or normal pigs. All tests for PRRS virus, pseudorabies virus and other cytopathic viruses were negative. PCV was demonstrated by in situ hybridization, PCR and IFA testing in tissues in 13 of 13 CT pigs and in 5 of 6 clinically normal pigs. Central nervous tissues and liver were the tissues most commonly infected with PCV in both CT and clinically normal pigs. In situ hybridization demonstrated PCV in PCV-positive pigs in the central nervous tissues of 12/13 CT and ⅚ clinically normal pigs, in the liver of 11/13 CT and 2/6 clinically normal pigs and in a lower proportion of all other tissues in CT and ⅚ clinically normal pigs. Few scattered cells that were morphologically typical of macrophages were positive in liver and other non-nervous tissues. PCV nucleic acid was only in the cytoplasm of most positive macrophages and in the nuclei of few positive macrophages. There were more PCV-positive cells in the central nervous tissues of both CT and clinically normal pigs than in other tissues. Positive cells in the brain and spinal cord were predominantly large neurons with fewer positive small neurons and rare positive oligodendrocytes. Large neurons in cerebral and medullar nuclei were positive, Purkinje cells in the cerebellum were positive and large neurons in the spinal gray matter were positive, especially lower motor neurons. Like macrophages, positive neurons usually had PCV nucleic acid only in the cytoplasm and rarely in the nucleus.

PCV infected cells in the central nervous system were more numerous and more widely distributed (Table 5) in CT pigs than in clinically normal pigs. Generally, CT pigs had large numbers of positive, large neurons diffusely distributed in the brain and spinal cord. Clinically normal pigs had fewer PCV-positive, large neurons distributed multifocally in the brain and spinal cord.

TABLE 5
Proportion of sampling sites positive for PCV by in-situ hybridization in 1-2
day-old PCV-infected pigs that had congenital tremors (CT) or were clinically normal.
No. positive/No. sampling sites examined

| | | | | Cervical | Thoracic | Lumbo- | Total |
|---|---|---|---|---|---|---|---|
| | Cerebrum | Cerebellum | Medulla | cord | cord | sacral | cord |
| CT | 9/13 | 9/13 | 8/13 | 20/33 | 20/44 | 17133 | 83/149 |
| (N = 13) | (.69) | (.69) | (.62) | (.61) | (.45) | (.52) | (.56) |
| Normal* | 1/5 | 3/5 | 2/5 | 1/15 | 1/20 | 3/15 | 11/65 |
| (N = 5) | (.20) | (.60) | (.40) | (.07) | (.05) | (.20) | (.17) |

*only the 5 clinically normal pigs that were PCV positive are included in the table, one clinically normal pig was negative for PCV with all test methods applied in this study

Indirect fluorescent antibody testing on frozen sections of cerebellum and liver from a single pig from each herd confirmed the results of in situ hybridization testing. PCV-specific antigens were demonstrated in approximately the same number and type of cells and in the same cellular locations as were PCV-specific nucleic acids with in situ hybridization. PCR testing of cerebellum and liver from all pigs demonstrated amplification of PCV2 specific sequences but not PCV1 specific sequences in all positive pigs from all 4 farms.

### Discussion

During outbreaks of CT type A2, both clinically normal and CT pigs were infected with PCV2 at 1-2 days-of-age. It is likely that both were born virus-infected. PCV2 was widely distributed in all infected pigs and was most common in central nervous tissues.

However, there were many more PCV-infected cells in the brain and spinal cord of CT pigs when compared to clinically normal pigs due to more diffuse distribution and a larger proportion of infected cells. The most commonly infected cells were large neurons in the brain and spinal cord and macrophages in non-neural tissues. Few oligodendrocytes were infected.

Previous studies have demonstrated that CT pigs are born with deficient myelin in the brain and spinal cord (Christensen, Nord Veterinaermed 8:921-943, 1956). Other studies demonstrated that CT pigs had abnormally immature myelin composed of disproportionately low cerebroside and high cholesterol esters relative to age-and-genetically matched normal control pigs (Patterson, J Neurochem 26:481-485, 1976). The hypothesis prior to this study was that oligodendrocytes would be the primary cell infected with PCV in the CNS accounting for reduced and abnormal myelin synthesis. The finding of large numbers of PCV-infected neurons in the brain and spinal cord was surprising and may be a significant cause of CT apart from, or in addition to, myelin deficiency. Previous studies determined that the degree of myelin deficiency in CT pigs was variable and not closely correlated with the severity of tremors (Fletcher, J Am Vet Med Assoc 29(12):2255-2262, 1968). This finding suggests that dysmyelinogenesis alone cannot account for CT. In one study, surgical ablations were performed on the nervous system of CT and control pigs including decerebration, unilateral labyrinthectomy, unilateral rhizotomy of lumbosacrat roots and transection of the thoracic spinal cord and it was determined by post-surgical neurological examinations that the cause of tremors is located at the spinal cord level. Others determined that the spinal reflex in CT pigs is monosynaptically hyperexcitable (Stromberg, Am J Vet Res 20:319-323, 1959). It may be that PCV infection of motor neurons in the spinal cord has a direct affect on function, rendering them more excitable and thus influencing spinal reflex arcs.

The paucity of PCV2-infected oligodendrocytes does not support the hypothesis of dysfunction of PCV2-infected oligodendrocytes as the cause of myelin deficiency in CT type A2. However, the inventors did not confirm myelin deficiency in these pigs, so it is possible that none existed. In addition, previous PCV-induced loss and removal of oligodendrocytes in these pigs cannot be ruled out. The granulomatous inflammatory reaction that is associated with PCV2 infection in pigs with PMWS was not observed in any PCV2-infected tissues of these CT pigs. The reason for a lack of inflammation is not clear, but in utero infection with PCV might induce immunotolerance.

### Example III

### Congenital Transmission of PCV2

### Methods

Eight pregnant high parity sows from a high-health-status commercial swine farm, seronegative to porcine respiratory and reproductive syndrome virus (PRRSV), were used to produce caesarian-derived/colostrums-deprived (CD/CD) pigs. All sows were moved into isolation rooms at Purdue University prior to 90 days of gestation and on day 94 of gestation 4 sows were inoculated intramuscularly and intranasally with 1.0 ml (107 TCID50/ml) of tissue culture-adapted PCV2 (PMWS-PCV-P4). Caesarian surgery followed by euthanasia was performed on the 4 PCV inoculated sows and 4 non-PCV-inoculated sows on day 114 of gestation to produce PCV2 congenitally exposed CD/CD pigs (C+pigs) and PCV2 congenitally free CD/CD pigs (C− pigs). Necropsy examinations were performed on sows and samples of serum, brain, spinal cord, liver, lung, spleen, tracheobronchial lymph node and inguinal lymph node were collected for testing for various porcine viruses. Three neonatal pigs from each litter were euthanized 3 days after birth. The remaining neonatal pigs were used in an experiment to produce lesions of PMWS. Complete necropsy examinations of euthanized pigs and pigs that died during the first 2 weeks after caesarian section were performed. Samples of brain, spinal cord, liver, lung, kidney, spleen, tonsil, bone marrow, thymus, tracheobronchial lymph node and inguinal lymph node were collected from each pig and preserved at −20° C. and or in neutral buffered formalin (except serum and blood). Histopathology and PCV in-situ hybridization were completed on all tissues to evaluate microscopic lesions and PCV distribution. PCV2 PCR was performed on selected pooled samples.

### Results

Fifteen pigs derived from 4 C+ sows died and 3 more pigs were euthanized during the first 2 weeks after caesarian section. Four pigs that died during the first 3 days and one additional piglet that was born dead, had dome shaped heads, were small, weak and had difficulties to orientate themselves in their environment. All other 11. C+ pigs lacked vigor and several died due to bacterial septicemia. Generally, C+ pigs lacked vigor and failed to thrive for the first 2 weeks after birth compared to C− pigs. No gross lesions characteristic of PMWS were identified in 19 C+pigs that were necropsied during the first 3 weeks of the study.

Microscopically, 8 C+ pigs that died during the first 3 weeks of age had a severe interstitial pneumonia suggesting of bacterial septicemia. No other microscopic lesions were found in these pigs. Sections of lymph nodes from 5 of the C+ pigs that died during the first 3 weeks of age, sections of liver from 1 pig, sections of lungs from 2 pigs, and sections of heart from 1 pig were suspect for PCV2 by in-situ hybridization. Pools of lymphoid tissues including spleen, tonsils, bronchial, and mesenteric lymph nodes from individual pigs were positive for PCV2 by PCV in 7 of 19C+ pigs. Samples of lung, spleen, and bronchial lymph node from all C+ pigs were negative by virus isolation and FA tests for pseudorabies virus (PRV), swine influenza virus (SIV), porcine reproductive and respiratory syndrome virus (PRRSV), porcine parvovirus (PPV) or transmissible gastroenteritis virus (TGEV). The available blood samples from 3 of the C+ pigs that bled during the first 3 weeks of age were serologically negative for PCV2 and for PPV. The PCV2 inoculum was found to be contaminated with PPV.

### Discussion

These results suggest PCV2 can be transmitted from an infected sow to its litter. PCV2 alone or in combination of a co-factor could be congenitally transmitted.

### Example IV

### Raising PCV-Specific Antibodies in Rabbits

The open reading frames (ORFs) representing PMWS-PCV-P1 ORF1 (314R), PMWS-PCV-P1 ORF2 carboxy region (129R), CT-PCV-P6 ORF3 (104R), CT-PCV-P6 ORF4 (59R), CT-PCV-P7 ORF2 (carboxy part) were amplified by PCR using suitable primers. PCR products were inserted into a bacterial expression and purification vector pET30a that drives the expression of the foreign insert fused to the histidine cassette. Expression of PCV proteins in bacteria was very efficient suggesting that these ORFs have the potential to code for functional proteins. PCV-specific proteins as fusion proteins were purified by Ni++ affinity chromatography. These purified proteins were used to immunize rabbits to raise antibodies.

### Example V

### Development of ELISA to Screen for PCV2 Antibody

The inventors have more particularly developed an ELISA assay for screening pig sera to detect antibody against PCV2 by using bacterially expressed PMWS-PCV-P1 ORF2 carboxy-portion purified protein to coat the plates.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

All patents, patent applications, publications, and other materials cited herein are hereby incorporated herein reference in their entireties.

What is claimed is:

1. An isolated nucleic acid from a porcine circovirus (PCV), which nucleic acid has a sequence that is identical to a sequence selected from the group consisting of SEQ ID NO. 1 to SEQ ID NO. 7.

2. A nucleic acid comprising the isolated nucleic acid of claim 1.

3. An isolated nucleic acid from porcine circovirus (PCV), which nucleic acid comprises a sequence coding for a circovirus polypeptide having a sequence selected from the group consisting of sequences coded by any of ORF1 to ORF11 of any of the sequences of SEQ ID NO. 1 to SEQ ID NO. 7.

4. The nucleic acid of claim 3 having a nucleotide sequence selected from the group consisting of sequences ORF1 to ORF11 of any of the sequences of SEQ ID NO. 1 to SEQ ID NO. 7.

5. An expression vector comprising the nucleic acid of claim 3 operatively associated with an expression control sequence.

6. A vaccine comprising the expression vector of claim 5 and a pharmaceutically acceptable excipient.

7. A host cell comprising the expression vector of claim 5.

8. A method for producing a PCV protein, which method comprises culturing a host cell of claim 7 under conditions that result in expression of the nucleic acid coding for a circovirus protein.

9. The method according to claim 8, wherein the PCV protein has an amino acid sequence selected from the group consisting of sequences coded by any of ORF1 to ORF11 of sequences added by any of the sequences of SEQ ID NO. 1 to SEQ ID ORF1 to ORF11 of sequences added by any of the sequences of SEQ ID NO. 1 to SEQ ID NO. 7.

10. The isolated polypeptide from a porcine circovirus (PCV) which has a sequence selected from the group consisting of sequences coded by any of ORF1 to ORF11 of any of the sequences of SEQ ID NO. 1 to SEQ ID NO. 7.

11. A vaccine comprising the isolated polypeptide of claim 10 and an adjuvant.

12. An isolated porcine circovirus strain, which has a genome comprising a sequence selected from the group consisting of sequences of SEQ ID NO. 1 to SEQ ID NO. 6.

13. The porcine circovirus of claim 12 which is attenuated, inactivated, or killed.

14. A vaccine comprising a porcine circovirus of claim 13 and an adjuvant.

15. A method of diagnosing a pathological cause of congenital tremors in a pig, which method comprises determining whether the pig has been infected by a porcine circovirus strain of type 1 or type 2.

16. The method according to claim 15, wherein the porcine circovirus has a genome comprising a sequence selected from the group consisting of sequences SEQ ID NO:1 to SEQ ID NO:6.

17. A method of diagnosing a pathological cause of congenital tremors in a pig, which method comprises determining whether the pig has been infected by a porcine circovirus strain has a genome comprising a sequence selected from the group consisting of sequences SEQ ID NO; 1 to SEQ ID NO:6.

18. The method according to claim 17, wherein the determination of the infection is effected by detecting the presence of a PCV nucleic acid in a biological sample from the pig.

19. The method according to claim 18, which comprises detecting hybridization of an oligonucleotide of at least about 20 bases that has a sequence found in 20 contiguous bases of SEQ ID NO: 1 to SEQ ID NO:6 or a complement thereof.

20. The method according to claim 17, wherein the determination of the infection is effected by detecting the presence of a PCV polypeptide in a biological sample from the pig.

21. The method according to claim 20, which comprises detecting binding of an antibody that specifically binds a polypeptide which has a sequence selected from the group consisting of sequences coded by any of ORF1 to ORF11 of any of the sequences of SEQ ID NO:1 to SEQ ID NO:6.

22. The method according to claim 17, wherein the determination of the infection is effected by detecting the presence of antibodies directed against a PCV polypeptide in a biological sample of the pig.

23. An antibody directed against the polypeptide of claim 10.

24. A method for the prevention or treatment of congenital tremors in a pig or its progeny, which method comprises administering to a pig in need of such treatment an immunoprotective amount of a vaccine comprising an immunogenic polypeptide of a type 1 or type 2 PCV strain and an adjuvant.

25. The method according to claim 24 wherein the PCV polypeptide has an amino acid sequence selected from the group consisting of sequences coded by any of ORF1 to ORF11 of any of the sequences of SEQ ID NO:1 to SEQ ID NO:6.

26. A method for the prevention or treatment of congenital tremors in a pig or its progeny, which method comprises administering to a pig in need of such treatment an immunoprotective amount of a vaccine comprising an immunogenic polypeptide that has an amino acid sequence selected from the group consisting of sequences coded by any of ORF1 to ORF11 of any of the sequences of SEQ ID NO:1 to SEQ ID NO:6 and an adjuvant.

27. A method for the prevention or treatment of congenital tremors in a pig or its progeny, which method comprises administering to a pig in need of such treatment an immunoprotective amount amount of a PCV nucleic acid that encodes an immunogenic polypeptide of a type 1 or type 2 PCV strain, with a pharmaceutically acceptable carrier.

28. The method according to claim 27 wherein the PCV polypeptide has an amino acid sequence selected from the group consisting of sequences coded by any of ORF1 to ORF11 of any of the sequences of SEQ ID NO:1 to SEQ ID NO:6.

29. A method for the prevention or treatment of congenital tremors in a pig or its progeny, which method comprises administering to a pig in need of such treatment an immunoprotective amount amount of a PCV nucleic acid that encodes an immunogenic polypeptide that has an amino acid sequence selected from the group consisting of sequences coded by any of ORF1 to ORF11 of any of the sequences of SEQ ID NO:1 to SEQ ID NO:6 with a pharmaceutically acceptable carrier.

30. A method for the prevention or treatment of congenital tremors in a pig or its progeny, which method comprises administering to a pig in need of such treatment an immunoprotective amount of an antibody of claim 23.

31. A method of culturing a porcine circovirus strain, which method comprises introducing a nucleic acid comprising a sequence selected from the group consisting of SEQ ID NO:1 to SEQ ID NO:6 into a suitable host cell under conditions that result in the production of porcine circovirus particles having a genome that comprises a sequence selected from the group consisting of SEQ ID NO:1 to SEQ ID NO:6.

32. The method according to claim 31, wherein the host cell is a PK-15 cell.

33. The method of claim 31, wherein the nucleic acid is introduced in the form of a cloned double-stranded DNA.
